(19) United States
(12) Patent Application Publication
(10) Pub. No.: US 2021/0023143 A1
(43) Pub. Date: Jan. 28, 2021

(54) METHODS FOR ELEVATION OF LIPID AND CHOLESTEROL METABOLISM

(71) Applicant: Minovia Therapeutics Ltd., Tirat Hacarmel (IL)

(72) Inventors: Natalie Yivgi Ohana, Haifi (IL); Uriel Halavee, Tel Aviv (IL); Shmuel Bukshpan, Ramat Hasharon (IL)

(21) Appl. No.: 17/042,038

(22) PCT Filed: Mar. 26, 2019
(86) PCT No.: PCT/IL2019/050350

§ 371 Date: Sep. 25, 2020

Related U.S. Application Data
(60) Provisional application No. 62/648,398, filed on Mar. 27, 2018.

(51) Int. Cl. A61K 35/50; A61P 3/06; A61P 3/04

(52) U.S. Cl. CPC A61K 35/50; A61K 9/0053; A61P 3/04; A61P 3/06

(57) ABSTRACT

The present invention relates to methods of using compositions comprising intact mitochondria and/or ruptured mitochondria for elevating lipid metabolism in cells. The present invention further provides methods for treating diseases which benefit from elevation of lipid and cholesterol metabolism and methods for inducing weight loss or reducing weight gain comprising administering compositions comprising intact mitochondria and/or ruptured mitochondria to a subject in need thereof.

### FIELD OF THE INVENTION

The present invention relates to methods of using compositions comprising intact mitochondria and/or ruptured mitochondria for elevating lipid metabolism in cells. The present invention further provides methods for treating diseases which benefit from elevation of lipid and cholesterol metabolism and methods for inducing weight loss or reducing weight gain comprising administering compositions comprising intact mitochondria and/or ruptured mitochondria to a subject in need thereof.

### BACKGROUND OF THE INVENTION

Mitochondria perform numerous essential tasks in the eukaryotic cell such as pyruvate oxidation, the Krebs cycle and metabolism of amino acids, fatty acids and steroids. The primary function of mitochondria is the generation of energy as adenosine triphosphate (ATP) by means of the electron-transport chain and the oxidative-phosphorylation system (the “respiratory chain”). Additional processes in which mitochondria are involved include heat production, storage of calcium ions, calcium signaling, programmed cell death (apoptosis) and cellular proliferation. Mitochondria are found in nearly all eukaryotes and vary in number and location depending on the cell type. Mitochondria contain their own DNA (mtDNA) and their own machinery for synthesizing RNA and proteins. mtDNA have only 37 genes, thus most of the gene products in the mammalian body are encoded by nuclear DNA.

WO 2013/035101 to the present inventors relates to mitochondrial compositions and therapeutic methods of using same, and discloses compositions of partially purified functional mitochondria and methods of using the compositions to treat conditions which benefit from increased mitochondrial function by administering the compositions to a subject in need thereof.

Pathological conditions that affect storage, breakdown and intestinal absorption of lipids are included in a broad category of so-called “lipid metabolism disorders”. Lipid metabolism disorders include: diet-induced and regular hypercholesterolemia, abetalipoproteinemia and hypobetalipoproteinemia. Several other lipid metabolism disorders of unknown origin have also been identified, including Anderson's disease and atherosclerosis. General symptoms of lipid metabolism disorders include, but are not limited to, chronic diarrhea, inadequate weight gain or weight loss, obesity and inability to lose excess weight.

U.S. Pat. No. 6,616,926 is directed to methods of modulating lipid metabolism and storage.

U.S. Pat. No. 6,929,806 is directed to agents for improving lipid metabolism and reducing high blood pressure. A milk-derived basic protein fraction and a basic peptide fraction are provided for use as an effective component for agents for improving lipid metabolism and reducing high blood pressure.

U.S. Pat. No. 7,238,727 provides compositions for improving lipid metabolism, compositions for preventing or treating hyperlipemia, compositions for preventing or treating obesity and foods for preventing or ameliorating hyperlipemia and obesity, which contain valine as an active ingredient.

Nakamura et al. (Journal of food science and technology, 53(1), 581-590; 2016) provide characterization of bioactive agents in five types of marketed sprouts and comparison of their antihypertensive, antihyperlipidemic, and antidiabetic effects in fructose-loaded spontaneously hypertensive rats (SHRs). There remains an unmet medical need for new therapeutic methods for reducing lipid and cholesterol levels in subjects in need thereof as well as methods for effective treatment of obesity, the methods being safe, cost effective, with minimal side effects and low to moderate invasiveness.

### SUMMARY OF THE INVENTION

The present invention, in embodiments thereof, discloses, for the first time, methods for elevating lipid and cholesterol metabolism by administration of a composition comprising intact mitochondria and/or ruptured mitochondria to a subject in need thereof. In particular, the present invention discloses methods for decreasing the lipid content in cells, thereby inducing reduction in body fat in a subject in need thereof. According to some embodiments, the present invention discloses methods of reducing levels of total and/or low density lipoprotein (LDL) cholesterol in a subject in need thereof. Each possibility represents a separate embodiment of the present invention.

The present invention is based in part on the unexpected discovery that incubation of adipocytes with mitochondria results in a significant decrease in cellular lipid accumulation, as exemplified herein below.

According to one aspect, the present invention provides a composition comprising intact mitochondria and/or ruptured mitochondria for use in elevating lipid and cholesterol metabolism in a subject in need thereof.

According to another aspect, the present invention provides a composition comprising intact mitochondria and/or ruptured mitochondria for use in inducing weight loss or attenuating or reducing weight gain in a subject in need thereof.

According to another aspect, the present invention provides a method for elevating lipid and cholesterol metabolism in a subject in need thereof, said method comprising:
(a) providing a composition comprising intact mitochondria and/or ruptured mitochondria; and
(b) administering to the subject a therapeutically effective amount of the composition, thereby elevating lipid or cholesterol metabolism.

According to yet another aspect, the present invention provides a method for inducing weight loss or attenuating or reducing weight gain in a subject in need thereof, said method comprising:
(a) providing a composition comprising intact mitochondria and/or ruptured mitochondria; and
(b) administering to said subject a therapeutically effective amount of the composition.

According to some embodiments, the composition comprising the ruptured mitochondria further comprises at least one mitochondrial constituent released from the ruptured mitochondria. According to some embodiments, the at least one mitochondrial constituent is selected from the group consisting of: mitochondrial protein, mitochondrial nucleic acid, mitochondrial lipid, mitochondrial peptide, mitochondrial saccharide, mitochondrial structure, at least part of a mitochondrial matrix and a combination thereof. Each possibility represents a separate embodiment of the present invention.

According to some embodiments, the composition comprising the intact mitochondria further comprises a hypertonic solution. According to further embodiments, the hypertonic solution comprises a saccharide. According to yet further embodiments, the hypertonic solution comprises sucrose.

According to some embodiments, the mitochondria are isolated mitochondria, wherein the weight of the mitochondrial proteins in the isolated mitochondria constitutes more than 80% of the combined weight of the mitochondria and other sub-cellular cellular proteins.

According to other embodiments, the mitochondria are partially purified mitochondria, wherein the weight of the mitochondrial proteins in the partially purified mitochondria constitutes between 10%-80% of the combined weight of the mitochondria and other sub-cellular proteins. According to specific embodiments, the weight of the mitochondrial proteins in the partially purified mitochondria constitutes between 20%-40% of the combined weight of the mitochondria and other sub-cellular proteins.

According to some embodiments, the mitochondria have undergone a freeze-thaw cycle.

According to some embodiments, the mitochondria are derived from a cell or a tissue selected from the group consisting of: placenta, placental cells grown in culture, blood cells, plant tissue, plant cells or plant cells grown in culture. Each possibility represents a separate embodiment of the present invention. According to specific embodiments, the mitochondria are derived from mung beans sprouts.

According to some embodiments, elevating lipid and cholesterol metabolism includes lowering of least one parameter selected from the group consisting of: blood concentration of total cholesterol, blood concentration of LDL cholesterol, blood concentration of triglycerides, concentration of fatty acids and/or triglycerides in adipose cells, or any combination thereof. Each possibility represents a separate embodiment of the present invention.

According to some embodiments, the composition is administered to the subject in need thereof by a route selected from the group consisting of: enteral, parenteral, intravenous, intraarterial, subcutaneous, oral and via direct injection into a tissue or an organ. Each possibility represents a separate embodiment of the present invention. According to specific embodiments, the composition is administered by oral administration. According to other embodiments, composition is administered into the adipose tissue of the subject.

According to some embodiments, the compositions and methods of the invention may be used for treating or preventing a disease which benefits from elevation of lipid and cholesterol metabolism.

According to some embodiments, the disease which benefits from elevation of lipid and cholesterol metabolism is selected from the group consisting of: obesity, a disease associated with increase in intraperitoneal adipose tissue, visceral obesity, visceral adipose tissue syndrome, fatty liver disease and cellulite. Each possibility represents a separate embodiment of the present invention. According to specific embodiments, said disease which benefits from elevation of lipid and cholesterol metabolism is obesity.

According to some embodiments, the methods of the invention further include administering a pharmacotherapy, wherein said pharmacotherapy is selected from the group consisting of: drugs that reduce fat absorption, drugs that regulate satiety, drugs for reducing the level of total and LDL cholesterol and any combination thereof. Each possibility represents a separate embodiment of the present invention.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a bar graph showing lipid content in 3T3-L1 cells differentiated into adipocytes and incubated with increasing amounts of mitochondria (“Mitos”), as evaluated using Oil-Red staining. Control=Un-differentiated 3T3-L1 cells, C+50 μl M=Un-differentiated 3T3-L1 cells incubated with 50 μl of mitochondria.

FIG. 2 is a bar graph comparing cholesterol content in bovine serum incubated with mitochondria (BS+Mito) or without mitochondria (BS).

FIG. 3 is a dot-plot showing 0₂ consumption over time in fresh (“Fresh”) vs. frozen mitochondria (“N2/-70”, flash frozen in liquid nitrogen and kept at −70° C. for 30 minutes). S=presence of 25 mM Succinate, S+ADP=presence of 25 mM Succinate and 1.65 mM ADP.

FIGS. 4A and 4B show dot plots comparing oxygen consumption of mitochondria incubated in isolation buffer (4A) or PBS (4B). FIG. 4C is a bar graph comparing citrate synthase release (%) from mitochondria incubated in isolation buffer or PBS.

FIG. 5A shows a dot plot comparing oxygen consumption of mitochondria incubated in isolation buffer or OptiMEM medium (Gibco); FIG. 5B is a bar graph comparing citrate synthase release from mitochondria incubated in isolation buffer or OptiMEM medium (Gibco).

FIG. 6 is a dot-plot showing O₂ consumption over time in a mitochondria composition comprising 20 mM sucrose (MP) or 200 mM sucrose (M).

FIG. 7 is a dot-plot showing O₂ consumption over time in mouse 3T3 cells treated with mitochondria suspended in isolation buffer (IB) or PBS (PBS).

FIG. 8 is a bar graph comparing citrate synthase activity of human 143B cells treated with mitochondria suspended in PBS (PBS) or mitochondria suspended in PBS that were frozen and thawed prior to treatment (PBS Frozen).

FIG. 9A is a dot-plot comparing mitochondrial O₂ consumption over time of mouse placental mitochondria suspended either in isolation buffer (“IB”) or PBS in the presence of succinate (S) or succinate+ADP (S+A); and FIG. 9B is a bar graph comparing citrate synthase release of mitochondria suspended in PBS or isolation buffer (IB).

FIG. 10 is a bar graph comparing citrate synthase activity in human 143B cells incubated with mitochondria suspended in either isolation buffer (IB) or PBS. NT=control, non-treated cells.

FIG. 11A is a dot-plot showing the change in body weight of C57BL mice fed with either high fat diet (HFD) or regular diet (reg), following treatment with low or high dose of mitochondria; and

FIG. 11B is a bar graph comparing the cholesterol levels in the HFD group vs. high dose mitochondria HFD group.

### DETAILED DESCRIPTION OF THE INVENTION

The present invention relates to compositions and methods for elevating metabolism of lipids and cholesterol in a subject in need thereof through administration of a composition comprising intact mitochondria and/or ruptured mitochondria. The present invention further provides compositions and methods for inducing weight loss or attenuating/reducing weight gain and for treatment or prevention of diseases which may benefit from elevation of lipid and cholesterol metabolism.

According to one aspect, the present invention provides a method for elevating lipid and cholesterol metabolism in a subject in need thereof, said method comprising:
(a) providing a composition comprising intact mitochondria and/or ruptured mitochondria; and
(b) administering to a subject in need thereof a therapeutically effective amount of the composition, thereby elevating lipid or cholesterol metabolism.

According to another aspect, the present invention provides a method for treating or preventing a disease which benefits from elevation of lipid and cholesterol metabolism, the method comprising:
(a) providing a composition comprising intact mitochondria and/or ruptured mitochondria; and
(b) administering to the subject a therapeutically effective amount of the composition.

According to yet another aspect, the present invention provides a method for inducing weight loss or attenuating or reducing weight gain in a subject in need thereof, the method comprising:
(a) providing a composition comprising intact mitochondria and/or ruptured mitochondria; and
(b) administering to the subject in need thereof a therapeutically effective amount of the composition, thereby inducing weight loss or attenuating/reducing weight gain in a subject.

According to another aspect, the present invention provides a method for inducing weight loss in a subject in need thereof, the method comprising:
(a) providing a composition comprising intact mitochondria and/or ruptured mitochondria; and
(b) administering to the subject in need thereof a therapeutically effective amount of the composition, thereby inducing weight loss in a subject.

According to another aspect, the present invention provides a method for attenuating or reducing weight gain in a subject in need thereof, the method comprising:
(a) providing a composition comprising intact mitochondria and/or ruptured mitochondria; and
(b) administering to the subject in need thereof a therapeutically effective amount of the composition, thereby attenuating/reducing weight gain in a subject.

According to one aspect, the present invention provides a composition comprising intact mitochondria and/or ruptured mitochondria for use in elevating lipid and cholesterol metabolism in a subject in need thereof.

According to another aspect, the present invention provides a composition comprising intact mitochondria and/or ruptured mitochondria for use in treating or preventing a disease which benefits from elevation of lipid and cholesterol metabolism.

According to yet another aspect, the present invention provides a composition comprising intact mitochondria and/or ruptured mitochondria for use in inducing weight loss or attenuating or reducing weight gain in a subject in need thereof.

According to another aspect, the present invention provides a composition comprising intact mitochondria and/or ruptured mitochondria for use in inducing weight loss in a subject in need thereof.

According to yet another aspect, the present invention provides a composition comprising intact mitochondria and/or ruptured mitochondria for use in attenuating or reducing weight gain in a subject in need thereof. According some aspects, the present invention provides a composition comprising intact mitochondria and/or ruptured mitochondria for use in attenuating weight gain in a subject in need thereof. According other aspects, the present invention provides a composition comprising intact mitochondria and/or ruptured mitochondria for use in reducing weight gain in a subject in need thereof.

According to some embodiments, the composition comprising the ruptured mitochondria further comprises at least one mitochondrial constituent released from the ruptured mitochondria. According to some embodiments, the mitochondrial constituent is selected from the group consisting of: mitochondrial protein, mitochondrial nucleic acid, mitochondrial lipid, mitochondrial saccharide, mitochondrial structure, at least part of a mitochondrial matrix and a combination thereof. Each possibility represents a separate embodiment of the present invention.

According to some embodiments, the composition comprising the intact mitochondria further comprises a hypertonic solution. According to some embodiments, the hypertonic solution comprises a saccharide. According to some embodiments, the hypertonic solution comprises sucrose.

According to another embodiment, the mitochondria have undergone a freeze-thaw cycle.

According to another embodiment, the composition is administered to the subject in need thereof by a route selected from the group consisting of: enteral, parenteral, intravenous, intraarterial, subcutaneous, oral and via direct injection into a tissue or an organ. Each possibility represents a separate embodiment of the present invention.

According to another embodiment, the composition is administered into the adipose tissue of the subject.

According to yet another embodiment, the composition may be administered by oral administration. According to another embodiment, the composition may be administered orally as a food additive. According to another embodiment, the composition may be administered orally as a food supplement. According to further embodiments, the composition may be administered as an additive to beverage or drink.

According to another embodiment, the mitochondria of the invention are derived from a cell or a tissue selected from the group consisting of: placenta, placental cells grown in culture and blood cells. According to another embodiment, the mitochondria of the invention are derived from a cell or a tissue selected from the group consisting of: human placenta, human placental cells grown in culture and human blood cells. According to yet another embodiment, the mitochondria of the invention are derived from plants. According to some embodiments, the mitochondria of the invention are derived from a plant tissue, plant cells or plant cells grown in culture. According to some embodiments, the mitochondria of the invention are derived from beans. According to specific embodiments, the mitochondria of the invention are derived from mung beans. According to further specific embodiments, the mitochondria of the invention are derived from mung bean sprouts.

According to another embodiment, the disease which benefits from elevation of lipid and cholesterol metabolism is selected from the group consisting of: obesity, a disease associated with increase in intraperitoneal adipose tissue, visceral obesity, visceral adipose tissue syndrome, fatty liver disease and cellulite. According to another embodiment, the disease which benefits from elevation of lipid and cholesterol metabolism is obesity.

According to another embodiment, the method of the invention further comprises administering an additional therapy. According to another embodiment, the additional therapy is selected from the group consisting of: dietary therapy, physical activity, behavioral therapy, pharmacotherapy and a combination thereof. Each possibility represents a separate embodiment of the present invention.

According to another embodiment, the pharmacotherapy may be selected from the group consisting of: drugs that reduce fat absorption, drugs that regulate satiety, drugs for reducing the level of total and LDL cholesterol and a combination thereof. Each possibility represents a separate embodiment of the present invention.

According to another embodiment, the drugs for reducing the level of total and LDL cholesterol are selected from the group consisting of: HMG CoA reductase inhibitors, nicotinic acid, fibric acid derivatives, bile acid sequestrants, cholesterol absorption inhibitors and combinations thereof. Each possibility represents a separate embodiment of the present invention.

It is to be understood that the term “elevating lipid and cholesterol metabolism” refers to any of the following options: elevating lipid metabolism, elevating cholesterol metabolism and a combination thereof.

According to some embodiments, the invention provides a method for elevating lipid and cholesterol metabolism. According to some embodiments, the invention provides a method for elevating lipid metabolism. According to some embodiments, the invention provides a method for elevating cholesterol metabolism. According to some embodiments, the invention provides a method for elevating at least one of: lipid metabolism and cholesterol metabolism.

According to some embodiments, the present invention provides a method for elevating lipid metabolism in a subject in need thereof, the method comprising: providing a composition comprising intact mitochondria and/or ruptured mitochondria; and administering to a subject in need thereof a therapeutically effective amount of the composition. Each possibility represents a separate embodiment of the present invention.

According to some embodiments, the present invention provides a method for treating a disease which benefits from elevation of lipid metabolism, the method inlucdes: providing a composition comprising intact mitochondria and/or ruptured mitochondria; and administering to a subject in need thereof a therapeutically effective amount of the composition. Each possibility represents a separate embodiment of the present invention.

As used herein, the term “elevating lipid metabolism” refers to increase in lipid metabolism. According to some embodiments, lipid metabolism refers to lipid metabolism within cells. According to some embodiments, elevating lipid metabolism includes, but is not limited to, reducing lipid content. According to some embodiments, elevating lipid metabolism is increasing lipid oxidation.

According to some embodiments, the term “lipid” refers to any type of lipid present in adipocytes. According to some embodiments, the term “lipid” refers to fatty acids, triglycerides and a combination thereof. Each possibility represents a separate embodiment of the present invention. The term “reducing lipid content” as used herein refers to decrease in the amount/concentration of fatty acids and/or triglycerides located in adipose cells (also referred to herein as adipocytes), thus reducing lipid levels (also referred to herein as lipid storage) in the adipocytes.

According to some embodiments, the term “elevating lipid metabolism” refers to reducing the amount/concentration of fatty acids and/or triglycerides located in adipose cells and/or in the blood, by a rate of at least 2%, 5%, 10%, 20%, 30%, 40%, 50% or 100%, as compared to the amount/concertation before treatment. The amount of fatty acids and/or triglycerides can be measured by methods known in the art, for example oil red 0 staining, titrimetric procedures of total lipid and enzymatic spectrophotometric procedures.

According to some embodiments, the present invention provides a method for reducing lipid content in a subject in need thereof, the method comprising: providing a composition comprising intact mitochondria and/or ruptured mitochondria; and administering to a subject in need thereof a therapeutically effective amount of the composition.

According to some embodiments, the term “elevating cholesterol metabolism” as used herein includes, but is not limited to, lowering blood concentration of total and/or low density lipoprotein (LDL) cholesterol. Each possibility represents a separate embodiment of the present invention. According to specific embodiments, the term “elevating cholesterol metabolism” refers to lowering blood concentration of total and/or low density lipoprotein (LDL) cholesterol or total cholesterol, by a rate of at least 2%, 5%, 10%, 15%, 20%, 30%, 40%, 50% or 100%, as compared to the concertation before treatment. According to some embodiments, the term “cholesterol” refers to total cholesterol. According to some embodiments, the term “cholesterol” refers to LDL cholesterol. According to some embodiments, the term “cholesterol” refers to total and/or LDL cholesterol. Each possibility represents a separate embodiment of the present invention. According to some embodiments, elevating cholesterol metabolism according to the methods of the invention treats and/or ameliorates high blood serum cholesterol. Each possibility represents a separate embodiment of the present invention.

The terms “high blood serum cholesterol” or “high total and LDL cholesterol” are used herein interchangeably and refer to cholesterol blood serum levels in a subject that are above the normal level present in a healthy subject. High blood serum cholesterol may lead to the development of a disease associated with high cholesterol in the serum. Normal levels of cholesterol vary between species and age groups. Typically, cholesterol is measured in a subject as either total plasma cholesterol, low density lipoprotein (LDL) cholesterol, high density lipoprotein (HDL) cholesterol and a combination thereof. Each possibility represents a separate embodiment of the present invention.

The terms “above the normal” and “higher than normal” with respect to cholesterol level are used interchangeably herein. Normal cholesterol level is dependent on various factors and can be determined according to health care providers standards. Typically, in an adult human, high blood serum cholesterol concentration is generally considered to be above about 5.2 to about 6.18 mmol/L (200-239 mg/dL) for total plasma cholesterol; and/or above about 3.36 to about 4.11 mmol/L (130-159 mg/dL) for LDL cholesterol. Lower limit of cholesterol level is considered healthy for a subject, depending on various factors such as the age and sex. For a child or adolescent, healthy cholesterol level is between about 120 mg/dL and about 170 mg/dL for total plasma cholesterol. In some embodiments, higher than normal cholesterol levels in a human subject is above about 240 mg/dL, above about 220 mg/dL, above about 200 mg/dL, above about 190 mg/dL, above about 180 mg/dL, or above about 170 mg/dL for total plasma cholesterol. Each possibility represents a separate embodiment of the present invention.

According to some embodiments, the present invention provides a method for elevating the metabolism of total and/or LDL cholesterol in a subject in need thereof, the method comprising: providing a composition comprising intact mitochondria and/or ruptured mitochondria; and administering to a subject in need thereof a therapeutically effective amount of the composition. Each possibility represents a separate embodiment of the present invention. According to some embodiments, the present invention provides a method for elevating the metabolism of total and/or LDL cholesterol in a subject suffering from high total and LDL cholesterol. Each possibility represents a separate embodiment of the present invention. According to some embodiments, the present invention provides a method for elevating the metabolism of total and/or LDL cholesterol in a healthy subject. Each possibility represents a separate embodiment of the present invention.

According to some embodiments, the present invention provides a method for reducing the serum level of total and/or LDL cholesterol in a subject in need thereof, the method comprising: providing a composition comprising intact mitochondria and/or ruptured mitochondria; and administering to a subject in need thereof a therapeutically effective amount of the composition. Each possibility represents a separate embodiment of the present invention. According to some embodiments, the present invention provides a method for reducing the serum level of total and/or LDL cholesterol in a subject suffering from high total and LDL cholesterol. Each possibility represents a separate embodiment of the present invention. According to some embodiments, the present invention provides a method for reducing the serum level of total and/or LDL cholesterol in a healthy subject. Each possibility represents a separate embodiment of the present invention.

According to some embodiments, the methods of the invention are used for treating or preventing a disease which benefits from elevation of lipid and/or cholesterol metabolism. Each possibility represents a separate embodiment of the present invention. As used herein, “a disease which benefits from elevation of lipid and/or cholesterol metabolism” refers to a disease resulting in levels of lipid and/or cholesterol which are higher than normal or a disease which may be aggravated by levels of lipid and/or cholesterol which are higher than normal. Each possibility represents a separate embodiment of the present invention. According to another embodiment, a disease which benefits from elevation of lipid and/or cholesterol metabolism is a disease associated with excess lipid storage. It is to be noted that normal levels of lipid and/or cholesterol are relative to patient parameters such as, but not limited to, sex and age. According to some embodiments, a disease which benefits from elevation of lipid and/or cholesterol metabolism is cellulite. Each possibility represents a separate embodiment of the present invention. As used herein, the term “cellulite” refers to herniation of subcutaneous lipid from within fibrous connective tissue.

According to another embodiment, a disease which benefits from elevation of lipid and/or cholesterol metabolism is selected from the group consisting of: obesity, a disease associated with increase in intraperitoneal adipose tissue, visceral obesity, visceral adipose tissue syndrome, fatty liver disease and cellulite. Each possibility represents a separate embodiment of the present invention. According to another embodiment, a disease which benefits from elevation of lipid and/or cholesterol metabolism is obesity. Without wishing to be bound by any theory or mechanism, administration of the composition of the invention to a subject afflicted with a disease which benefits from elevation of lipid and/or cholesterol metabolism results in reduction of lipid and/or cholesterol levels and thus in treatment or amelioration of the disease. It is to be understood that the methods of the present invention may treat a subject suffering from a disease which benefits from elevation of lipid and/or cholesterol metabolism or a subject susceptible to or suspected of having a disease which benefits from elevation of lipid and/or cholesterol metabolism. Each possibility represents a separate embodiment of the present invention.

As used herein, unless otherwise specified, the term “preventing a disease” includes, but is not limited to, inhibition or the averting of symptoms associated with a particular disease or disorder. As used herein, unless otherwise specified, the term “treating” refers to the administration of the composition after the onset of symptoms of the disease or disorder whereas “preventing” refers to the administration prior to the onset of the symptoms, particularly to patients at risk of the disease or disorder.

According to some embodiments, the compositions and methods of the invention are used to induce weight loss in a subject. According to some embodiments, the compositions and methods of the invention are used to induce reduction in body fat in a subject. Without wishing to be bound by any theory or mechanism, administration of the composition of the invention results in elevation of lipid and/or cholesterol metabolism, thus reducing body fat and inducing weight loss in a subject.

According to some embodiments, the compositions and methods of the invention can induce weight loss or attenuate or reduce weight gain in a subject that otherwise would not have lose weight or attenuate or reduce weight gain under similar conditions (i.e. under the same lifestyle, diet or physical activity).

According to some embodiments, the composition and methods of the invention are used to attenuate or reduce weight gain in a subject. According to some embodiments, the composition and methods of the invention are used to prevent weight gain in a subject. According to other embodiments, the compositions and methods of the invention are used to prevent, attenuate or reduce weight regain in a subject. According to further embodiments, the compositions and methods of the invention are used to prevent, attenuate or reduce weight gain in a subject susceptible to become overweight/obese. According to specific embodiment, the compositions and methods of the invention are used for preventing, attenuating or reducing weight gain associated with type 2 diabetes, drug treatment, smoking cessation and the like. Each possibility represents a separate embodiment of the present invention.

As used herein, the terms “attenuate or reduce weight gain” and “attenuating or reducing weight gain” refer to diminishing the increase in weight of a patient. The terms “attenuate or reduce weight regain” and “attenuating or reducing weight regain” refer to diminishing the increase in weight of a patient experiencing rebound in weight after weight loss. Weight regain may be due to a rebound effect following cessation of weight loss achieved via diet, exercise, behavior modification, or approved therapies.

It is to be understood that the compositions and methods of the present invention may be employed for the treatment of overweight/obese individuals and/or for the treatment of subjects susceptible to become overweight/obese. According to some embodiments, the compositions and methods of the invention are further directed to treat subjects having topical lipid storage disorders (also known as lipidoses) that do not fall under the definition of obesity or overweight. According to some embodiments, the compositions and methods of the present invention may be used for medical weight loss as well as for non-medical weight loss.

As used herein, the term “subject in need thereof” refers to a subject inflicted or in risk of being inflicted with a disease which benefits from elevation of lipid and/or cholesterol metabolism. According to some embodiments, a subject in need thereof is a subject who requires or desires weight loss or a subject needing lower lipid and/or cholesterol levels. Each possibility represents a separate embodiment of the present invention. According to some embodiments, the term “subject in need thereof” refers to a subject suffering from at least one of the conditions selected from group consisting of: obesity, overweight, visceral obesity, a disease or a disorder associated with excess lipid storage, a disease associated with increase in intra peritoneal adipose tissue, visceral adipose tissue syndrome, fatty liver disease, cellulite and a combination thereof.

Each possibility represents a separate embodiment of the invention. As used herein, the term “subject” may refer to any human or non-human subjects. In one embodiment, the subject is a mammalian subject. In specific embodiments, the mammalian subject is a human subject.

In some embodiments, the subject may be a human subject inflicted or in risk of being inflicted with a disease which benefits from elevation of lipid and/or cholesterol metabolism; a human subject who requires or desires weight loss or a human subject needing lower lipid and/or cholesterol levels. Each possibility represents a separate embodiment of the invention. In other embodiments, the subject is a human subject suffering from at least one of the conditions selected from group consisting of: obesity, overweight, visceral obesity, a disease or a disorder associated with excess lipid storage, a disease associated with increase in intra peritoneal adipose tissue, visceral adipose tissue syndrome, fatty liver disease, cellulite and a combination thereof. Each possibility represents a separate embodiment of the invention.

The term “overweight” as used herein, refers to a body mass index (BMI) of 25 to 29.9kg/m². The term “obese” as used herein refers to a BMI of >30 kg/m². It is to be noted that the scope of the terms “overweight” and “obese” may be evaluated by any weight evaluation method known in the art, and is not limited to evaluation based on the body mass index.

The term “visceral obesity” as used herein refers to a form of obesity due to excessive deposition of fat in the abdominal viscera and omentum, rather than subcutaneously.

The term “therapeutically effective amount” as used herein refers to the amount of the composition of the invention effective to elevate lipid and/or cholesterol metabolism. Each possibility represents a separate embodiment of the present invention. According to some embodiments, the term “therapeutically effective amount” as used herein refers to the amount of the composition of the invention effective to elevate lipid and/or cholesterol metabolism to a level which enables treating or preventing a disease which benefits from elevation of lipid and/or cholesterol metabolism. Each possibility represents a separate embodiment of the present invention. According to other embodiments, the term “therapeutically effective amount” as used herein refers to the amount of the composition of the invention effective to elevate lipid and/or cholesterol metabolism to a level which enables achieving weight loss or reducing/preventing/attenuating weight gain in a subject. Each possibility represents a separate embodiment of the present invention.

The term “therapeutically effective amount” with specific reference to cholesterol metabolism refers to the amount of composition effective to reduce the total and/or LDL cholesterol in the blood of a subject in need thereof to a level which is not considered high. Each possibility represents a separate embodiment of the present invention.

According to another embodiment, the method of the invention further comprises administering an additional therapy. According to another embodiment, the additional therapy is selected from the group consisting of: dietary therapy, physical activity, behavioral therapy, pharmacotherapy and a combination thereof. Each possibility represents a separate embodiment of the present invention. According to another embodiment, the pharmacotherapy is selected from the group consisting of: drugs that reduce fat absorption, drugs that regulate satiety, drugs for reducing the level of total and LDL cholesterol and a combination thereof. Each possibility represents a separate embodiment of the present invention.

As used herein, the terms “pharmacotherapy for reducing total and LDL cholesterol” and “drugs for reducing the level of total and LDL cholesterol” are used interchangeably. According to another embodiment, the pharmacotherapy for reducing total and LDL cholesterol is selected from the group consisting of: 3-hydroxy-3-methyl-glutaryl- CoA reductase (HMG CoA reductase) inhibitors, nicotinic acid, fibric acid derivatives, bile acid sequestrants, cholesterol absorption inhibitors and a combination thereof. Each possibility represents a separate embodiment of the present invention.

As used herein, the term “the composition” and “the composition of the invention” are used interchangeably. According to some embodiments, the term “the composition of the invention”, as used herein, refers to a composition comprising intact and/or ruptured mitochondria. According to some embodiments, the term “the composition of the invention”, as used herein, refers to mitochondria selected from the group consisting of: intact mitochondria and ruptured mitochondria. According to some embodiments, the composition of the invention comprises ruptured mitochondria. According to some embodiments, the composition of the invention comprises intact mitochondria. According to other embodiments, the composition of the invention comprises intact mitochondria and ruptured mitochondria. According to some embodiments, the composition of the invention comprises at least one mitochondrial constituent. According to some embodiments, the composition of the invention comprises ruptured mitochondria and at least one mitochondrial constituent. According to some embodiments, the composition of the invention comprises ruptured mitochondria and at least one mitochondrial constituent released and/or secreted from the ruptured mitochondria. Each possibility represents a separate embodiment of the present invention. According to some embodiments, the composition of the invention comprises partially purified mitochondria. According to some embodiments, the composition of the invention comprises isolated mitochondria. According to some embodiments, the composition of the invention comprises a medium conditioned by mitochondria. According to other embodiments, the composition of the invention comprises at least one of the group consisting of: ruptured mitochondria, at least one mitochondrial constituent, isolated mitochondria, partially purified mitochondria, intact mitochondria, a media conditioned by mitochondria and a combination thereof. Each possibility represents a separate embodiment of the present invention. As used herein, the term “medium conditioned by mitochondria” refers to a medium in which mitochondria were incubated and which contains mitochondrial constituents and/or elements secreted from mitochondria.

As used herein, the terms “composition comprising intact mitochondria and/or ruptured mitochondria”, “composition comprising mitochondria selected from the group consisting of: intact mitochondria and ruptured mitochondria” and “composition comprising mitochondria” may interchangeably be used. The terms are directed to a composition which comprises intact mitochondria, ruptured mitochondria, or a combination of both intact and ruptured mitochondria.

Mitochondria include the mitochondrial genome which is a circular double-stranded molecule, consisting of 16,569 base pairs. It contains 37 genes including 13 protein-encoding genes, 22 transfer RNA (tRNA) genes and two ribosomal RNA (rRNA) genes. The 13 protein-encoding genes are components of the mitochondrial respiratory chain. The wild type (wt)-mtDNA molecule may also include sequence polymorphism, but it remains fully functional. Structurally, mitochondria organelles range in diameter or width from 0.5 μm to 1 μm and have four compartments: the outer membrane, the inner membrane, the intermembrane space and the matrix.

As used herein, the terms “mitochondria” or “the mitochondria of the invention” are interchangeable and refer to intact mitochondria and/or ruptured mitochondria. According to some embodiments, the mitochondria of the invention refer to intact mitochondria. According to some embodiments, the mitochondria of the invention refer to ruptured mitochondria. According to some embodiments, the mitochondria of the invention refer to ruptured mitochondria and at least one mitochondrial constituent secreted or released from the mitochondria. Each possibility represents a separate embodiment of the present invention. According to some embodiments, the mitochondria of the invention refer to mitochondria from which at least one mitochondrial constituent is released according to the present invention.

The mitochondria according to the invention may be obtained by methods disclosed herein or by any other method known in the art. Commercially available mitochondria isolation kits include, for example Mitochondria Isolation Kit, MITOISO1 (Sigma-Aldrich), among others.

According to some embodiments, the mitochondria of the invention are functional mitochondria. According to another embodiment, partially purified mitochondria are functional mitochondria. According to another embodiment, the mitochondria of the invention are not functional. According to another embodiment, the mitochondria of the invention are isolated mitochondria. According to another embodiment, the mitochondria of the invention are intact mitochondria. According to another embodiment, the mitochondria of the invention are partially-functional. As used herein, partially-functional mitochondria refer to mitochondria lacking at least one functional property of mitochondria, such as, but not limited to, oxygen consumption. According to some embodiments, ruptured mitochondria are non-functional mitochondria. According to some embodiments, ruptured mitochondria are partially-functional mitochondria.

According to some embodiments, the term “functional mitochondria” refers to mitochondria that consume oxygen. According to another embodiment, functional mitochondria have an intact outer membrane. According to some embodiments, functional mitochondria are intact mitochondria. According to some embodiments, functional mitochondria consume oxygen at an increasing rate over time. According to some embodiments, the functionality of mitochondria is measured by oxygen consumption. According to some embodiments, oxygen consumption of mitochondria may be measured by any method known in the art such as, but not limited to, the MitoXpress fluorescence probe (Luxcel). According to some embodiments, functional mitochondria are mitochondria which display an increase in the rate of oxygen consumption in the presence of ADP and a substrate such as, but not limited to, glutamate, malate or succinate. Each possibility represents a separate embodiment of the present invention. According to some embodiments, functional mitochondria are mitochondria which produce ATP. According to some embodiments, functional mitochondria are mitochondria capable of manufacturing their own RNAs and proteins and are self-reproducing structures. According to some embodiments, functional mitochondria produce a mitochondrial ribosome and mitochondrial tRNA molecules.

As is known in the art, functional placental mitochondria participate in production of progesterone (see, for example, Tuckey R C, Placenta, 2005, 26(4):273-81). According to some embodiments, functional mitochondria are mitochondria which produce progesterone or pregnenolone. Each possibility represents a separate embodiment of the present invention. According to some embodiments, functional mitochondria are mitochondria which secrete progesterone. In a non-limiting example, mitochondria derived from placenta or placental cells grown in culture produce progesterone or pregnenolone. Each possibility represents a separate embodiment of the present invention. According to some embodiments, the mitochondria of the invention are derived from placenta or placental cells grown in culture and the mitochondria produce progesterone or pregnenolone. Each possibility represents a separate embodiment of the present invention. According to some embodiments, the production of progesterone or pregnenolone in the intact mitochondria of the invention is not impaired following a freeze-thaw cycle. According to some embodiments, the functionality of mitochondria is measured by measuring mitochondrial progesterone production or mitochondrial production of progesterone precursors such as, but not limited to, pregnenolone. Each possibility represents a separate embodiment of the present invention. Progesterone production may be measured by any assay known in the art such as, but not limited to, a radioimmunoassay (RIA).

As used herein, the term “derived” when in reference to mitochondria relates to the source from which the mitochondria were obtained. For example, the source may be cells or tissue selected from the group consisting of: placenta, placental cells grown in culture, blood cells, plant tissue, plant cells and plant cells grown in culture.

As used herein, the term “partially purified mitochondria” refers to mitochondria separated from other cellular components, wherein the weight of the mitochondria constitutes between 10-80%, 20-80%, 20-70%, 40-70%, 20-40%, or 20-30% of the combined weight of the mitochondria and other sub-cellular fractions (as exemplified in: Hartwig et at, Proteomics, 2009, (9):3209-3214). Each possibility represents a separate embodiment of the present invention. According to another embodiment, partially purified mitochondria do not contain intact cells.

According to another embodiment, the weight of the mitochondrial proteins in partially purified mitochondria constitutes at least 10% of the combined weight of the mitochondria and other sub-cellular proteins. According to another embodiment, the weight of the mitochondrial proteins in partially purified mitochondria constitutes at least 20% of the combined weight of the mitochondria and other sub-cellular proteins. According to another embodiment, the weight of the mitochondrial proteins in partially purified mitochondria constitutes between 10%-80% of the combined weight of the mitochondria and other sub-cellular proteins. According to another embodiment, the weight of the mitochondrial proteins in partially purified mitochondria constitutes between 20%-80% of the combined weight of the mitochondria and other sub-cellular proteins. According to another embodiment, the weight of the mitochondrial proteins in partially purified mitochondria constitutes between 20%-40% of the combined weight of the mitochondria and other sub-cellular proteins. According to yet another embodiment, the weight of the mitochondrial proteins in partially purified mitochondria constitutes between 20%-30% of the combined weight of the mitochondria and other sub-cellular proteins. According to another embodiment, the weight of the mitochondrial proteins in partially purified mitochondria constitutes between 40%-80% of the combined weight of the mitochondria and other sub-cellular proteins. According to another embodiment, the weight of the mitochondrial proteins in partially purified mitochondria constitutes between 30%-70% of the combined weight of the mitochondria and other sub-cellular proteins. According to another embodiment, the weight of the mitochondrial proteins in partially purified mitochondria constitutes between 50%-70% of the combined weight of the mitochondria and other sub-cellular proteins. According to another embodiment, the weight of the mitochondrial proteins in partially purified mitochondria constitutes between 60%-70% of the combined weight of the mitochondria and other sub-cellular proteins. According to another embodiment, the weight of the mitochondrial proteins in partially purified mitochondria constitutes less than 80% of the combined weight of the mitochondria and other sub-cellular proteins.

As used herein, the term “mitochondrial proteins” refers to proteins which originate from mitochondria, including mitochondrial proteins which are encoded by genomic DNA or mtDNA. As used herein, the term “sub-cellular proteins” refers to all proteins which originate from the cells or tissue from which the mitochondria are produced.

As used herein, the term “isolated mitochondria” refers to mitochondria separated from other cellular components, wherein the weight of the mitochondrial proteins constitutes more than 80% of the combined weight of the mitochondria and other sub-cellular cellular proteins. Preparation of isolated mitochondria may require changing buffer composition or additional washing steps, cleaning cycles, centrifugation cycles and sonication cycles which are not required in preparation of partially purified mitochondria. Without wishing to be bound by any theory or mechanism, such additional steps and cycles may harm the functionality of the isolated mitochondria.

According to one embodiment, the weight of the mitochondrial proteins in isolated mitochondria constitutes more than 80% of the combined weight of the mitochondria and other sub-cellular cellular proteins. According to another embodiment, the weight of the mitochondrial proteins in isolated mitochondria constitutes more than 90% of the combined weight of the mitochondria and other sub-cellular proteins. A non-limiting example of a method for obtaining isolated mitochondria is the MACS® technology (Miltenyi Biotec). Without wishing to be bound by any theory or mechanism, isolated mitochondria in which the weight of the mitochondria constitutes more than 95% of the combined weight of the mitochondria and other sub-cellular fractions are not functional mitochondria. According to another embodiment, isolated mitochondria do not contain intact cells. According to some embodiments, the mitochondria of the invention are isolated mitochondria.

As used herein, the term “intact mitochondria” refers to mitochondria comprising an outer membrane, an inner membrane, the cristae (formed by the inner membrane) and the matrix. According to some embodiments, intact mitochondria comprise mitochondrial DNA. As used herein, the term “mitoplasts” refers to mitochondria devoid of outer membrane. According to another embodiment, intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Each possibility represents a separate embodiment of the present invention. Mitochondria that were observed under a microscope and show TMRM or TMRE staining have an intact mitochondrial outer membrane. According to some embodiments, intactness of a mitochondrial membrane is measured by assaying the presence of citrate synthase outside mitochondria. According to some embodiments, mitochondria that release citrate synthase have compromised mitochondrial intactness. According to some embodiments, intactness of a mitochondrial membrane is determined by measuring the mitochondrial rate of oxygen consumption coupled to presence of ADP. According to some embodiments, an increase in mitochondrial oxygen consumption in the presence of ADP is indicative of an intact mitochondrial membrane. According to some embodiments, intact mitochondria according to the invention are partially purified mitochondria. According to some embodiments, intact mitochondria according to the invention are isolated mitochondria. According to some embodiments, functional mitochondria are intact mitochondria.

As used herein, the term “a mitochondrial membrane” refers to a mitochondrial membrane selected from the group consisting of: the mitochondrial inner membrane, the mitochondrial outer membrane or a combination thereof.

As used herein, the term “ruptured mitochondria” refers to mitochondria in which the inner and outer mitochondrial membranes have been sheared (torn), perforated, punctured and the like. According to some embodiments, ruptured mitochondria are mitochondria that have been sheared to more than one piece/portion. It is to be understood that ruptured mitochondria are intact mitochondria that had been ruptured by the methods described herein or any other method known in the art.

According to some embodiments, ruptured mitochondria are mitochondria that released at least one mitochondrial constituent from the mitochondria. According to some embodiments, ruptured mitochondria are directed to mitochondria in which the inner and outer mitochondrial membranes have been torn, perforated, punctured and the like and which released at least one mitochondrial constituent. According to some embodiments, rupture of intact mitochondria results in release of at least one mitochondrial constituent. It is to be understood that, according to some embodiments, ruptured mitochondria that have released at least one mitochondrial constituent are administered together with the released constituent.

As used herein, the term “mitochondrial constituent” refers to any element comprised in mitochondria. According to some embodiments, a mitochondrial constituent is at least one element selected from the group consisting of: mitochondrial protein, mitochondrial peptide, mitochondrial nucleic acid, mitochondrial lipid, mitochondrial saccharide, mitochondrial structure, at least part of a mitochondrial matrix and a combination thereof. Each possibility represents a separate embodiment of the present invention.

As used herein, the term “mitochondrial structure” refers to structures and/or organelles present in mitochondria, such as, but not limited to, matrix granules, ATP-synthase particles, mitochondrial ribosomes and cristae. According to some embodiments, a mitochondrial constituent maintains at least one function of intact functional mitochondria. According to some embodiments, a mitochondrial constituent comprises a single type of mitochondrial protein, mitochondrial peptide (e.g., Humanin), mitochondrial nucleic acid, mitochondrial lipid, mitochondrial structure or mitochondrial saccharide. Each possibility represents a separate embodiment of the present invention. According to some embodiments, a mitochondrial constituent comprises at least one functioning protein. According to some embodiments, a mitochondrial constituent comprises at least part of the mitochondrial matrix. According to some embodiments, a mitochondrial constituent comprises the entire mitochondrial matrix. According to some embodiments, a mitochondrial constituent comprises at least part of the mitochondrial matrix and at least part of the elements comprised therein, such as, but not limited to proteins, adenosine triphosphate (ATP) or ions. According to some embodiments, a mitochondrial constituent comprises at least part of the mitochondrial matrix and at least one of the following elements comprised therein: mitochondrial protein, mitochondrial nucleic acid, mitochondrial lipid, mitochondrial saccharide and a mitochondrial structure. Each possibility represents a separate embodiment of the present invention. As used herein, the term “mitochondrial matrix” refers to the viscous material within the mitochondrial inner membrane.

It is to be understood that mitochondrial constituents according to some embodiments of the present invention are elements secreted or released from mitochondria, such as, but not limited to mitochondrial proteins. According to some embodiments, mitochondrial constituents which are secreted or released from mitochondria may be retrieved by any method known in the art, such as, but not limited to, retrieving the mitochondrial constituents from a conditioned medium in which mitochondria have been incubated.

According to some embodiments, mitochondrial constituents may be obtained by any method known in the art for isolation of mitochondria fractions from cells, for example, the method carried out by using the Mitochondria isolation kit for culture cells from Thermo Fisher Scientific (Rockford, Ill., USA). According to some embodiments, mitochondrial fractions or constituents are produced as a byproduct of mitochondria isolation or partial purification. Each possibility represents a separate embodiment of the present invention.

According to some embodiments, the present invention provides a method for elevating lipid and cholesterol metabolism in a subject in need thereof, the method comprising: providing a composition comprising at least one mitochondrial constituent; and administering to a subject in need thereof a therapeutically effective amount of the composition. According to some embodiments, the present invention provides a method for treating or preventing a disease which benefits from elevation of lipid and cholesterol metabolism, the method comprising: providing a composition comprising at least one mitochondrial constituent; and administering to the subject a therapeutically effective amount of the composition. According to some embodiments, the present invention provides a method for inducing weight loss in a subject in need thereof, the method comprising: providing a composition comprising at least one mitochondrial constituent; and administering to the subject in need thereof a therapeutically effective amount of the composition. According to other embodiments, the present invention provides a method for preventing, attenuating or reducing weight gain in a subject in need thereof, the method comprising: providing a composition comprising at least one mitochondrial constituent; and administering to the subject in need thereof a therapeutically effective amount of the composition. According to further embodiments, the present invention provides a method for preventing, attenuating or reducing weight regain in a subject in need thereof, the method comprising: providing a composition comprising at least one mitochondrial constituent; and administering to the subject in need thereof a therapeutically effective amount of the composition.

According to some embodiments, the present invention provides a composition comprising at least one mitochondrial constituent for use in elevating lipid and cholesterol metabolism in a subject in need thereof. According to some embodiments, the present invention provides a composition comprising at least one mitochondrial constituent for use in treating or preventing a disease which benefits from elevation of lipid and cholesterol metabolism. According to some embodiments, the present invention provides a composition comprising at least one mitochondrial constituent for use in inducing weight loss in a subject in need thereof. According to other embodiments, the present invention provides a composition comprising at least one mitochondrial constituent for use in attenuating or reducing weight gain in a subject in need thereof. According to other embodiments, the present invention provides a composition comprising at least one mitochondrial constituent for use in attenuating or reducing weight regain in a subject in need thereof.

It is to be understood that ruptured mitochondria and/or mitochondrial constituents according to some embodiments of the present invention are obtained from intact and/or isolated and/or partially purified mitochondria. Each possibility represents a separate embodiment of the present invention. It is to be further understood that mitochondrial constituents according to preferred embodiments of the present invention are obtained from intact mitochondria through any method known in the art. According to some embodiments, the mitochondrial constituents of the invention are obtained by transferring the intact mitochondria from a hypertonic solution to a hypotonic solution. According to some embodiments, transferring intact mitochondria from a hypertonic to a hypotonic solution results in release of at least one mitochondrial constituent. Each possibility represents a separate embodiment of the present invention.

As used herein, the terms “hypotonic”, “isotonic” and “hypertonic” relate to a concentration relative to the solute concentration inside intact mitochondria. According to other embodiments, ruptured mitochondria are obtained by exposing intact mitochondria to a hypotonic solution, such as, but not limited to, a hypotonic phosphate-buffered saline (PBS) solution. Without wishing to be bound by any theory or mechanism, exposing intact mitochondria to a hypotonic solution results in explosion or perforation of the mitochondria, thus obtaining ruptured mitochondria, possibly releasing mitochondrial constituents such as, but not limited to, at least part of the mitochondrial matrix.

According to some embodiments, ruptured mitochondria are obtained by transferring mitochondria from a hypertonic solution to a hypotonic solution. Without wishing to be bound by any theory or mechanism, transferring intact mitochondria from a hypertonic solution to a hypotonic solution results in explosion, rupture or perforation of the mitochondria, thus obtaining ruptured mitochondria, possibly releasing mitochondrial constituents such as, but not limited to, at least part of the mitochondrial matrix. In a non-limiting example, explosion, rupture or perforation of intact mitochondria may result in release of mitochondrial proteins such as citrate synthase. According to some embodiments, release of citrate synthase is used as an indication of ruptured mitochondria. According to some embodiments, mitochondrial constituents according to the present invention are released from intact mitochondria by increasing the osmotic pressure within the intact mitochondria. Without wishing to be bound by any theory or mechanism, increasing the osmotic pressure within intact mitochondria such that mitochondrial membranes are perforated and/or torn results in ruptured mitochondria and possibly in release of mitochondrial constituents according to the present invention.

According to some embodiments, a composition comprising intact mitochondria according to the present invention is formulated as a hypertonic solution. According to some embodiments, the composition of the invention comprises a hypertonic solution. According to some embodiments, a hypertonic solution according to the present invention comprises a saccharide.

As used herein the term “saccharide” may refer to a saccharide, an oligosaccharide or a polysaccharide. Each possibility represents a separate embodiment of the present invention. According to some embodiments, the saccharide is sucrose. According to some embodiments, the concentration of the saccharide in the hypertonic solution according to the present invention is similar to the concentration of the saccharide in the isolation buffer. According to some embodiments, a sufficient saccharide concentration which acts to preserve mitochondrial function is sufficient for preserving mitochondria intact. According to some embodiments, the isolation buffer is hypertonic. According to other embodiments, the saccharide concentration in the hypertonic solution, according to the present invention, is a sufficient saccharide concentration for preserving mitochondria intact. According to some embodiments, the composition of the invention further comprises a sufficient saccharide concentration for preserving mitochondria intact.

According to another embodiment, a sufficient saccharide concentration for preserving mitochondria intact is a concentration of between 100 mM-400 mM, preferably between 100 mM-250 mM, most preferably between 200 mM-250 mM. Each possibility represents a separate embodiment of the present invention. According to another embodiment, a sufficient saccharide concentration for preserving mitochondria intact is between 100 mM-150 mM. According to another embodiment, a sufficient saccharide concentration for preserving mitochondria intact is between 150 mM-200 mM. According to another embodiment, a sufficient saccharide concentration for preserving mitochondria intact is between 100 mM-200 mM. According to another embodiment, a sufficient saccharide concentration for preserving mitochondria intact is between 100 mM-400 mM. According to another embodiment, a sufficient saccharide concentration for preserving mitochondria intact is between 150 mM-400 mM. According to another embodiment, a sufficient saccharide concentration for preserving mitochondria intact is between 200 mM-400 mM. According to another embodiment, a sufficient saccharide concentration for preserving mitochondria intact is at least 100 mM. Without wishing to be bound by any theory or mechanism of action, a saccharide concentration below 100 mM may not be sufficient to preserve mitochondria intact. According to some embodiments, a saccharide concentration above 100 mM is hypertonic.

According to some embodiments, a composition comprising ruptured mitochondria according to the present invention is formulated as a hypotonic solution. According to some embodiments, the composition of the invention comprises a hypotonic solution. A non-limiting example of a hypotonic solution is Phosphate Buffered Saline (PBS). According to some embodiments, mitochondria in PBS are ruptured mitochondria. According to other embodiments, mitochondria in isolation buffer are intact mitochondria. According to some embodiments, mitochondria in an isolation buffer comprising a saccharide concentration sufficient for preserving mitochondria intact are intact mitochondria.

According to some embodiments, the intact mitochondria of the invention are exposed to an ion-exchanger inhibitor. According to some embodiments, the intact mitochondria of the invention are reduced in size by exposure to an ion-exchanger inhibitor. According to another embodiment, the intact mitochondria of the invention were reduced in size by exposure to an ion-exchanger inhibitor. According to some embodiments, the intact mitochondria of the invention are exposed to the ion-exchanger inhibitor following partial purification or isolation. Each possibility represents a separate embodiment of the present invention. According to some embodiments, the intact mitochondria of the invention are exposed to the ion-exchanger inhibitor during partial purification or isolation. Each possibility represents a separate embodiment of the present invention. According to other embodiments, the cells or tissue from which the intact mitochondria of the invention are derived are exposed to the ion-exchanger inhibitor prior to partial purification or isolation of the mitochondria. Each possibility represents a separate embodiment of the present invention. According to another embodiment, the ion-exchanger inhibitor is CGP37157. As used herein, the terms “CGP” and “CGP37157” are used interchangeably. Without wishing to be bound by any theory or mechanism, agents blocking the mitochondrial Na⁺/Ca²⁺ exchanger, such as, CGP37157 may induce mitochondrial fission, increase mitochondrial ATP production and reduce mitochondrial size. Mitochondrial fission refers to spontaneous fission or fission induced by appropriate agents such as CGP37157. According to another embodiment, the final composition of the invention is devoid of free ion-exchanger inhibitor. As used herein, a composition devoid of ion-exchanger inhibitor refers to a composition devoid of ion-exchanger inhibitor which is not bound to the mitochondria of the invention. According to some embodiments, the composition of the invention comprises an ion-exchanger inhibitor bound to the mitochondria of the invention. According to some embodiments, a composition devoid of ion-exchanger inhibitor comprises an ion-exchanger inhibitor at a concentration of less than 1 μM of, preferably less than 0.5 μM, most preferably less than 0.1 μM.

According to some embodiments, the mitochondria of the invention are derived from a different subject than the subject to whom they are administered. According to some embodiments, the mitochondria of the invention are derived from the same subject to whom they are administered. According to another embodiment, the mitochondria of the invention are from a source selected from allogeneic and xenogeneic. Each possibility represents a separate embodiment of the present invention. According to another embodiment, the mitochondria of the invention are from a source selected from syngeneic, allogeneic and xenogeneic. Each possibility represents a separate embodiment of the present invention. According to another embodiment, the mitochondria of the invention are derived from a cell or tissue from a source selected from allogeneic and xenogeneic. Each possibility represents a separate embodiment of the present invention. According to another embodiment, the mitochondria of the invention are derived from a cell or tissue from a source selected from syngeneic, allogeneic and xenogeneic. Each possibility represents a separate embodiment of the present invention.

As used herein, mitochondria of an allogeneic source refer to mitochondria derived from a different subject than the subject to be treated from the same species. As used herein, mitochondria of a xenogeneic source refer to mitochondria derived from a different subject than the subject to be treated from a different species. As used herein, the term “syngeneic” refers to genetically identical. According to some embodiments, an autologous cell is a syngeneic cell.

According to some embodiments, the mitochondria of the invention are derived from a mammalian subject. According to another embodiment, the mammalian subject is a human subject. According to another embodiment, the mitochondria of the invention are derived from a mammalian cell. According to another embodiment, the mammalian cell is a human cell. According to another embodiment, the mitochondria of the invention are derived from cells in culture. According to another embodiment, the mitochondria of the invention are derived from human cells in culture. According to another embodiment, the mitochondria of the invention are derived from a tissue.

According to some embodiments, the mitochondria of the invention are derived from a cell or a tissue selected from the group consisting of: human placenta, human placental cells grown in culture and human blood cells. Each possibility represents a separate embodiment of the present invention. According to another embodiment, the mitochondria of the invention are derived from a cell or a tissue selected from the group consisting of: placenta, placental cells grown in culture and blood cells. Each possibility represents a separate embodiment of the present invention.

According to some embodiments, the mitochondria of the invention are derived from a plant. According to some embodiments, the mitochondria of the invention are derived from a plant tissue, plant cells or plant cells grown in culture. Each possibility represents a separate embodiment of the present invention. According to some embodiments, deriving mitochondria from plant tissue, plant cells or plant cells grown in culture according to the present invention refers to deriving mitochondria from plant protoplasts. Plant mitochondria according to the present invention may be derived from any plant species, plant organ, plant cells or plant cells grown in culture known in the art to comprise mitochondria. Each possibility represents a separate embodiment of the present invention. In non-limiting examples, plant mitochondria according to the invention may be derived from storage organs (such as potato, sugar or beet), green leaves (such as tobacco, pea or petunia) or etiolated seedlings (such as wheat, maize or mung bean). According to specific embodiments, the mitochondria of the invention are derived from mung beans. According to some embodiments, the mitochondria of the invention are derived from mung beans sprouts. According to some embodiments, the mitochondria of the invention are derived from potato. According to some embodiments, the mitochondria of the invention are derived from algae, such as but not limited to, dunaliella. According to other embodiments, the mitochondria of the invention are obtained from an animal subject, preferably a mammalian subject, most preferably a human subject or human cells grown in culture. Each possibility represents a separate embodiment of the present invention. According to some embodiments, the mitochondria of the invention are obtained from cells lacking a cell wall, preferably mammalian cells, most preferably human cells. Each possibility represents a separate embodiment of the present invention.

According to some embodiments, ruptured mitochondria according to the present invention are derived from intact mitochondria. According to some embodiments, ruptured mitochondria according to the present invention are derived from intact partially purified mitochondria. According to some embodiments, ruptured mitochondria according to the present invention are derived from intact isolated mitochondria.

According to some embodiments, the intact and/or ruptured mitochondria of the invention are derived from a cell or a tissue selected from the group consisting of: human placenta, human placental cells grown in culture and human blood cells. Each possibility represents a separate embodiment of the present invention. According to another embodiment, the intact and/or ruptured mitochondria of the invention are derived from a cell or a tissue selected from the group consisting of: placenta, placental cells grown in culture and blood cells. Each possibility represents a separate embodiment of the present invention.

According to some embodiments, the mitochondrial constituent according to the present invention is produced from mitochondria derived from a cell or a tissue selected from the group consisting of: placenta, placental cells grown in culture and blood cells. Each possibility represents a separate embodiment of the present invention. According to some embodiments, the mitochondrial constituent according to the present invention is produced from mitochondria derived from a cell or a tissue selected from the group consisting of: human placenta, human placental cells grown in culture and human blood cells. Each possibility represents a separate embodiment of the present invention.

As used herein. the phrases “cells grown in culture” or “a tissue grown in culture” refers to a multitude of cells or a tissue, respectively, grown in a liquid, semi-solid or solid medium, outside of the organism from which the cells or tissue derive. According to some embodiments, cells grown in culture are cells grown in bioreactors. According to a non-limiting example, cells may be grown in a bioreactor (such as, but not limited to the bioreactor disclosed in WO 2008/152640), followed by isolation of partially purified functional mitochondria from the cells.

According to another embodiment, the mitochondria of the invention have undergone a freeze-thaw cycle. According to some embodiments, the intact mitochondria of the invention have undergone a freeze-thaw cycle. Without wishing to be bound by any theory or mechanism, intact mitochondria that have undergone a freeze-thaw cycle demonstrate at least comparable oxygen consumption rate following thawing, as compared to control intact mitochondria that have not undergone a freeze-thaw cycle. Thus, intact mitochondria that have undergone a freeze-thaw cycle are at least as functional as control mitochondria that have not undergone a freeze-thaw cycle.

As used herein, the term “freeze-thaw cycle” refers to freezing of the mitochondria of the invention to a temperature below 0° C., maintaining the mitochondria in a temperature below 0° C. for a defined period of time and thawing the mitochondria to room temperature or body temperature or any temperature above 0° C. which enables administration according to the methods of the invention. Each possibility represents a separate embodiment of the present invention. The term “room temperature”, as used herein refers to a temperature of between 18° C. and 25° C. The term “body temperature”, as used herein, refers to a temperature of between 35.5° C. and 37.5° C., preferably 37° C.

According to specific embodiment, the mitochondria of the invention have undergone lyophilization. According to further embodiments, the intact mitochondria of the invention have undergone lyophilization.

The term “lyophilization” as used herein is defined as a freeze drying or dehydration technique involving freezing the mitochondria of the invention and then reducing the concentration of one of the solvents, preferably a water miscible solvent, by sublimation and desorption, to levels which will no longer support biological or chemical reactions. This is usually accomplished by a drying step in a high vacuum.

According to some embodiments, the mitochondria that have undergone a freeze-thaw cycle were frozen at a temperature of at least −196° C. According to some embodiments, the mitochondria that have undergone a freeze-thaw cycle were frozen at a temperature of at least −70° C. According to some embodiments, the mitochondria that have undergone a freeze-thaw cycle were frozen at a temperature of at least −20° C. According to some embodiments, the mitochondria that have undergone a freeze-thaw cycle were frozen at a temperature of at least −4° C. According to some embodiments, the mitochondria that have undergone a freeze-thaw cycle were frozen at a temperature of at least 0° C. According to another embodiment, freezing of the mitochondria is gradual. According to some embodiment, freezing of mitochondria is through flash-freezing. As used herein, the term “flash-freezing” refers to rapidly freezing the mitochondria by subjecting them to cryogenic temperatures. In a non-limiting example, flash-freezing may include freezing using liquid nitrogen.

According to some embodiments, the mitochondria that underwent a freeze-thaw cycle were frozen for at least 30 minutes prior to thawing. According to another embodiment, the freeze-thaw cycle comprises freezing the mitochondria for at least 30, 60, 90, 120, 180, 210 minutes prior to thawing. Each possibility represents a separate embodiment of the present invention. According to some embodiments, the mitochondria that have undergone a freeze-thaw cycle were frozen for at least 1, 2, 3, 4, 5, 6, 7, 8, 9, 10, 24, 48, 72, 96, 120 hours prior to thawing. Each freezing time presents a separate embodiment of the present invention. According to some embodiments, the mitochondria that have undergone a freeze-thaw cycle were frozen for at least 4, 5, 6, 7, 30, 60, 120, 365 days prior to thawing. Each freezing time presents a separate embodiment of the present invention. According to another embodiment, the freeze-thaw cycle comprises freezing the mitochondria for at least 1, 2, 3 weeks prior to thawing. Each possibility represents a separate embodiment of the present invention. According to another embodiment, the freeze-thaw cycle comprises freezing the mitochondria for at least 1, 2, 3, 4, 5, 6 months prior to thawing. Each possibility represents a separate embodiment of the present invention.

According to some embodiments, the mitochondria that have undergone a freeze-thaw cycle were frozen at −70° C. for at least 30 minutes prior to thawing. Without wishing to be bound by any theory or mechanism, the possibility to freeze mitochondria and thaw them after a long period enables easy storage and use of the mitochondria with reproducible results even after a long period of storage. According to some embodiments, ruptured mitochondria according to the present invention are prepared/produced from intact mitochondria that have undergone a freeze-thaw cycle.

According to another embodiment, thawing is at room temperature. According to some embodiments, thawing is at body temperature. According to another embodiment, thawing is at a temperature which enables administration according to the methods of the invention. According to another embodiment, thawing is performed gradually.

As used herein, the term “isolation buffer” refers to a buffer in which the mitochondria of the invention have been partially purified or isolated. Each possibility represents a separate embodiment of the present invention. It is to be understood that intact mitochondria according to the invention are isolated or partially purified in isolation buffer, while ruptured mitochondria are produced from isolated/partially purified intact mitochondria by methods described herein or any other method known in the art. In a non-limiting example, the isolation buffer comprises 200 mM sucrose, 10 mM Tris-MOPS and 1 mM EGTA. According to some embodiments, BSA (Bovine Serum Albumin) is added to the isolation buffer during partial purification or isolation. Each possibility represents a separate embodiment of the present invention. According to some embodiments, 0.2% BSA is added to the isolation buffer during partial purification or isolation. Each possibility represents a separate embodiment of the present invention. According to some embodiments, HSA (Human Serum Albumin) is added to the isolation buffer during partial purification or isolation. Each possibility represents a separate embodiment of the present invention. According to some embodiments, 0.2% HSA is added to the isolation buffer during partial purification or isolation. Each possibility represents a separate embodiment of the present invention. According to other embodiment, HSA or BSA is washed away from the mitochondria of the invention following partial purification or isolation. Each possibility represents a separate embodiment of the present invention. Without wishing to be bound by any mechanism or theory, freezing mitochondria within the isolation buffer saves time and isolation steps, as there is no need to replace the isolation buffer with a freezing buffer prior to freezing or to replace the freezing buffer upon thawing.

According to another embodiment, the mitochondria that underwent a freeze-thaw cycle were frozen within a freezing buffer. According to another embodiment, the intact mitochondria that underwent a freeze-thaw cycle were frozen within the isolation buffer. According to another embodiment, the intact mitochondria that underwent a freeze-thaw cycle were frozen within a buffer comprising the same constituents as the isolation buffer.

According to another embodiment, the freezing buffer comprises a cryoprotectant. According to some embodiments, the cryoprotectant is a saccharide, an oligosaccharide or a polysaccharide. Each possibility represents a separate embodiment of the present invention. According to another embodiment, the saccharide concentration in the freezing buffer is a sufficient saccharide concentration which acts to preserve mitochondrial function. According to another embodiment, the isolation buffer comprises a saccharide. According to another embodiment, the saccharide concentration in the isolation buffer is a sufficient saccharide concentration which acts to preserve mitochondrial function. According to another embodiment, the saccharide concentration in the isolation buffer is a sufficient saccharide concentration which acts to keep mitochondria intact. According to another embodiment, the saccharide concentration in the freezing buffer is a sufficient saccharide concentration which acts to keep mitochondria intact. According to another embodiment, the saccharide is sucrose. Without wishing to be bound by any theory or mechanism, intact mitochondria that have been frozen within a freezing buffer or isolation buffer comprising sucrose demonstrate at least comparable oxygen consumption rate following thawing, as compared to control mitochondria that have not undergone a freeze-thaw cycle or that have been frozen within a freezing buffer or isolation buffer without sucrose.

According to some embodiments, ruptured mitochondria underwent a freeze-thaw cycle. According to some embodiments, a mitochondrial constituent according to the invention underwent a freeze-thaw cycle. According to some embodiments, the ruptured mitochondria that underwent a freeze-thaw cycle were frozen within a freezing buffer. According to some embodiments, the mitochondrial constituent that underwent a freeze-thaw cycle was frozen within a freezing buffer. According to some embodiments, the ruptured mitochondria that underwent a freeze-thaw cycle were frozen within a hypotonic solution, such as, but not limited to PBS. According to some embodiments, the mitochondrial constituent that underwent a freeze-thaw cycle was frozen within a hypotonic solution, such as, but not limited to PBS.

According to some embodiments, the ruptured mitochondria that underwent a freeze-thaw cycle were frozen within the isolation buffer. According to another embodiment, the ruptured mitochondria that underwent a freeze-thaw cycle were frozen within a buffer comprising the same constituents as the isolation buffer. According to some embodiments, the mitochondrial constituent that underwent a freeze-thaw cycle was frozen within the isolation buffer. According to another embodiment, the mitochondrial constituent that underwent a freeze-thaw cycle was frozen within a buffer comprising the same constituents as the isolation buffer.

According to some embodiments, ruptured mitochondria have undergone lyophilization. According to other embodiments, a mitochondrial constituent according to the invention underwent lyophilization.

Any suitable route of administration to a subject may be used for the composition of the present invention, including but not limited to local and systemic routes. According to some embodiments, administering is administering systematically. According to some embodiments, the composition is formulated for systemic administration. According to some embodiments, administering is administering locally. According to some embodiments, the composition is formulated for local administration.

According to another embodiment, administration systemically is through a parenteral route. According to another embodiment, administration locally is through a parenteral route. According to some embodiments, preparations of the composition of the invention for parenteral administration include sterile aqueous or non-aqueous solutions, suspensions, or emulsions, each representing a separate embodiment of the present invention. Non-limiting examples of non-aqueous solvents or vehicles are propylene glycol, polyethylene glycol, vegetable oils such as olive oil and corn oil, gelatin, and injectable organic esters such as ethyl oleate.

According to some embodiments, parenteral administration is administration intravenously, intra-arterially, intramuscularly, intraperitoneally, intradermally, transdermally or subcutaneously. Each of the abovementioned administration routes represents a separate embodiment of the present invention. According to another embodiment, parenteral administration is performed by bolus injection. According to another embodiment, parenteral administration is performed by continuous infusion. The preferred mode of administration will depend upon the particular indication being treated and will be apparent to one of skill in the art.

According to another embodiment, systemic administration of the composition is through injection. According to another embodiment, local administration of the composition is through injection. For administration through injection, the composition may be formulated in an aqueous solution, for example in a physiologically compatible buffer including but not limited to Hank's solution, Ringer's solution, or physiological salt buffer. Formulations for injection may be presented in unit dosage forms, for example, in ampoules, or in multi-dose containers with, optionally, an added preservative. According to another embodiment, administration is through convection enhanced delivery (CED).

Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles, and may contain formulatory agents such as suspending, stabilizing, and/or dispersing agents. Non-limiting examples of suitable lipophilic solvents or vehicles include fatty oils such as sesame oil, or synthetic fatty acid esters such as ethyl oleate or triglycerides.

According to another embodiment, the composition is administered intravenously, and is thus formulated in a form suitable for intravenous administration. According to another embodiment, the composition is administered intra-arterially, and is thus formulated in a form suitable for intra-arterial administration. According to another embodiment, the composition is administered intramuscularly, and is thus formulated in a form suitable for intramuscular administration.

According to another embodiment, administration systemically is through an enteral route. According to another embodiment, administration through an enteral route is oral administration. According to some embodiments, the composition is formulated for oral administration.

According to some embodiments, the composition is formulated for oral administration in a form of hard or soft gelatin capsules, pills, capsules, powders, tablets, including coated tablets, dragees, elixirs, suspensions, liquids, gels, slurries, syrups or inhalations and controlled release forms thereof. Each possibility represents a separate embodiment of the present invention.

Suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

If desired, disintegrating agents, such as cross-linked polyvinyl pyrolidone, agar, or alginic acid or a salt thereof, such as sodium alginate, may be added. Capsules and cartridges of, for example, gelatin for use in a dispenser may be formulated containing a powder mix of the composition of the invention and a suitable powder base, such as lactose or starch.

Solid dosage forms for oral administration include capsules, tablets, pill, powders, and granules. In such solid dosage forms, the composition of the invention is admixed with at least one inert pharmaceutically acceptable carrier such as sucrose, lactose, or starch. Such dosage forms can also comprise, as it normal practice, additional substances other than inert diluents, e.g., lubricating, agents such as magnesium stearate. In the case of capsules, tablets and pills, the dosage forms may also comprise buffering, agents. Tablets and pills can additionally be prepared with enteric coatings.

Liquid formulations for oral administration include solutions, emulsions, suspensions, syrups and the like, and may include conventional diluents such as water and liquid paraffin.

Liquid dosage forms for oral administration may further contain adjuvants, such as wetting agents, emulsifying and suspending agents, and sweetening, flavoring and perfuming agents, and preservatives. According to some embodiments, enteral coating of the composition is further used for oral or bucal administration. The term “enteral coating”, as used herein, refers to a coating which controls the location of composition absorption within the digestive system. Non-limiting examples for materials used for enteral coating are fatty acids, waxes, plant fibers or plastics.

In some embodiments, the composition of the invention may be included in a food or drink. These are, for example, yogurt, kefir, miso, natto, tempeh, kimchee, sauerkraut, water, milk, fruit juices, vegetable juices, carbonated soft drinks, non-carbonated soft drinks, coffee, tea, beer, wine, liquor, alcoholic mixed drinks, bread, cakes, cookies, crackers, extruded snacks, soups, frozen desserts, fried foods, pasta products, potato products, rice products, corn products, wheat products, dairy products, confectionaries, hard candies, nutritional bars, breakfast cereals, bread dough, bread dough mix, sauces, processed meats, and cheeses. Each possibility represents a separate embodiment of the present invention.

According to some embodiments, the composition of the invention may further comprise probiotics. According to specific embodiments, the composition of the invention is mixed with probiotics. Suitable examples of probiotics include, but are not limited to, lactobacillus and Bifidobacterium. According to further embodiments, the composition of the invention further comprises probiotics, wherein said composition is included in a food or a drink selected from the group consisting of: yogurt, kefir, miso, natto, tempeh, kimchee, sauerkraut, water, milk, fruit juices, vegetable juices, carbonated soft drinks, non-carbonated soft drinks, coffee, tea, beer, wine, liquor, alcoholic mixed drinks, bread, cakes, cookies, crackers, extruded snacks, soups, frozen desserts, fried foods, pasta products, potato products, rice products, corn products, wheat products, dairy products, confectionaries, hard candies, nutritional bars, breakfast cereals, bread dough, bread dough mix, sauces, processed meats, and cheeses, Each possibility represents a separate embodiment of the present invention.

The term “probiotics” refers to dietary supplements containing potentially beneficial bacteria or yeasts. According to the currently adopted definition by FAO/WHO, probiotics are: ‘Live microorganisms which when administered in adequate amounts confer a health benefit on the host’.

According to another embodiment, the composition of the invention is administered to a subject in need thereof by a route selected from the group consisting of: intravenous, intraarterial, subcutaneous, oral and via direct injection into tissue or an organ. Each possibility represents a separate embodiment of the present invention. For certain applications, such as treatment of gastrointestinal disorders or diseases, enteral administration may be feasible.

According to some embodiments, the composition of the invention is administered into the adipose tissue of a subject in need thereof. According to another embodiment, the composition of the invention is administered into the adipose tissue of a subject in need thereof locally. According to some embodiments, the composition of the invention is administered through injection into the adipose tissue of a subject in need thereof.

As used herein, the term “about”, when referring to a measurable value such as an amount, a temporal duration, and the like, is meant to encompass variations of +/−10%, or +/−5%, +/−1%, or even +/−0.1% from the specified value.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Example 1

### Bovine Placental Mitochondria Decrease Lipid Accumulation in 3T3-L1 Cells

Mitochondria were prepared from 400 mg bovine term placenta by the following protocol:
1. Placenta was rinsed free of blood by using ice-cold IB buffer (isolation buffer: 200 mM sucrose, 1 mM EGTA and 10 mM Tris-MOPS) +0.2% BSA.
2. The placenta was minced into small pieces in 5 ml IB+0.2% BSA using scissors.
3. The suspension was transferred to a (10 ml) glass potter and homogenized using a Dounce glass homogenizer by two complete up and down cycles.
4. The homogenate was transferred to a 15 ml tube and centrifuged at 600 g for 10 min at 4° C.
5. The supernatant was transferred to clean centrifuge tubes and the pellet resuspended in IB, and subjected to a second centrifugation step.
6. The supernatant was recovered, centrifuged at 7,000×g for 15 min.
7. The supernatant was discarded and the pellet resuspended in 10m1 ice-cold IB followed by centrifuge at 600g for 10 min at 4° C.
8. The mitochondria were recovered from the supernatant by centrifuging at 7,000×g for 15 min at 4° C.
9. The supernatant was discarded and the pellet resuspended, comprising mitochondria in 200 ul of IB.

10. The protein content was determined by the Bradford assay.

3T3-L1 cells were cultured in 24 well plates until confluent, with DMEM+10% fetal bovine serum, Dexametasone, insulin and IBMX according to the instruction of the

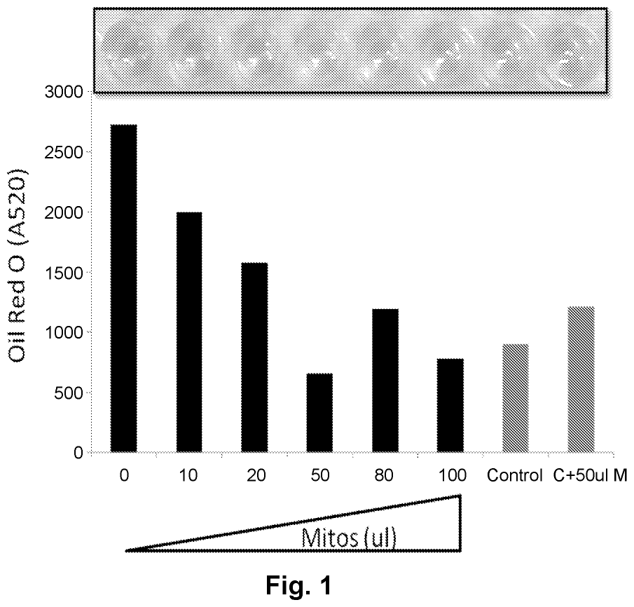

Adipogenesis kit (Chemicon). Cells were incubated with increasing amounts of mitochondria in 200 μl of differentiation medium for 24 hours. Cells were then washed in PBS and maintained in maintenance media for additional 5 days. Finally, the cells were stained with Oil-Red-O, lysed and the level of lipid accumulation was evaluated using a plate reader (520 nm) for each group. 3T3-L1 cells that were not treated with the adipogenesis kit (i.e. adipogenesis was not induced in said cells) were used as a control. In addition, Oil Red 0 quantification was measured in un-differentiated 3T3-L1 cells, incubated with 50 μl of mitochondria, serving as an additional control. The results, presented in FIG. 1, show that an increase in the amount of the mitochondria preparation was correlated with a decrease in the quantification of Oil Red O in the cells, indicative of a decrease in the lipid accumulation in the cells. Both control samples showed low levels of Oil Red O staining

### Example 2

### Bovine Placental Mitochondria Decrease Cholesterol in Bovine Serum

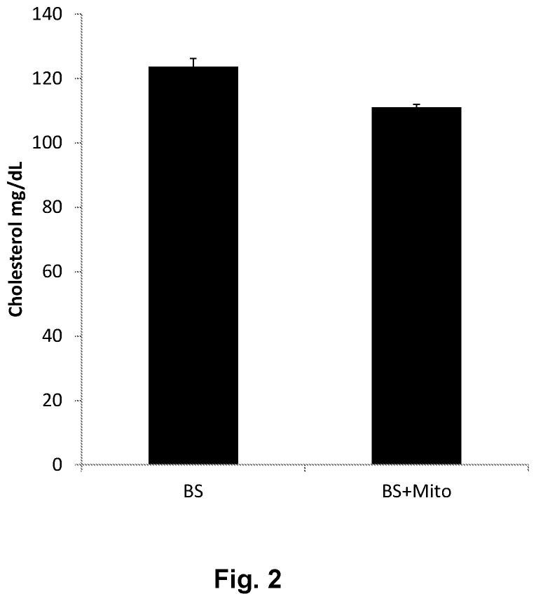

Bovine placental mitochondria were prepared as described in Example 1. Thirty micrograms of mitochondria were incubated for 24 h at 37° C. with 200 μl of bovine serum (Biological Industries, Israel). Following incubation, the levels of total Cholesterol in the serum were determined. As shown in FIG. 2, incubation with placental mitochondria induced a decrease of approximately 10% in serum cholesterol level.

### Example 3

### Mitochondria that were Frozen and Thawed Show Oxygen Consumption Comparable to that of Non-Frozen Mitochondria

Mitochondria were isolated from mouse term placenta according to the following protocol:
1. Placenta was rinsed free of blood by using ice-cold IB buffer (isolation buffer: 200 mM sucrose, 1 mM EGTA and 10 mM Tris-MOPS)+0.2% BSA.
2. The placenta was minced into small pieces in 5 ml IB+0.2% BSA using scissors.
3. The suspension was transferred to a 10 ml glass potter and homogenized using a Dounce glass homogenizer by five complete up and down cycles.
4. The homogenate was transferred to a 15 ml tube and centrifuged at 600 g for 10 min at 4° C.
5. The supernatant was transferred to clean centrifuge tubes and the pellet was resuspended in IB buffer, and subjected to a second centrifugation step.
6. The supernatant from steps 4 and 5 was filtered through a 5 μm filter to remove any cells or large cell debris.
7. The supernatant was recovered and centrifuged at 7,000×g for 15 min.
8. The mitochondrial pellet was washed in 10 ml ice cold IB buffer and mitochondria were recovered by centrifugation at 7,000×g for 15 min at 4° C.
9. The supernatant was discarded and the pellet resuspended, containing mitochondria in 200 μl of IB buffer.
10. Protein content was determined by the Bradford assay.

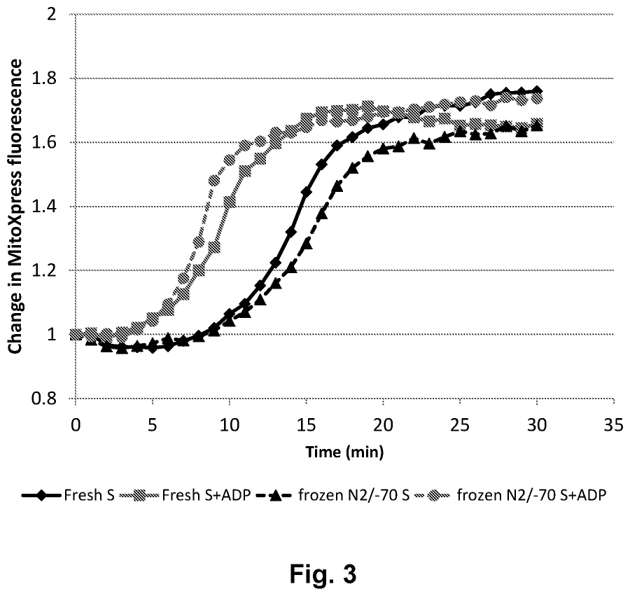

To compare activity of frozen versus unfrozen mitochondria, mitochondria were flash-frozen using liquid nitrogen in IB (200 mM sucrose, 1 mM EGTA and 10 mM Tris-MOPS) in 1.5 ml Eppendorf tubes and kept at −70° C. for 30 minutes. Mitochondria were thawed quickly by hand and O₂ consumption by 100 μg mitochondria was measured using the MitoXpress fluorescence probe (Luxcel) and a Tecan plate reader. Oxygen consumption was measured in the presence of 25 mM Succinate (S) or in the presence of 25 mM Succinate and 1.65 mM ADP (S+ADP). The change in fluorescence was calculated relative to the level of fluorescence at time 0. FIG. 3 shows that the O₂ consumption, and rate of O₂ consumption, were comparable for mitochondria that were frozen and thawed (marked “Frozen”) in comparison to non-frozen mitochondria (marked “Fresh”).

As opposed to frozen mitochondria, mouse placental mitochondria that were chilled (kept for 4 days at 4° C.) produced less ATP than fresh mitochondria (Table 1).

TABLE 1
ATP production of fresh and chilled mouse placental mitochondria

| | ATP (RLU) |
|---|---|
| Fresh Mitochondria (F) | 4690 |
| Chilled Mitochondria (C) | 1587 |

### Example 4

### Comparison of Oxygen Consumption and Membrane Integrity of Mitochondria Incubated in Isolation Buffer vs. Mitochondria Incubated in PBS

Mitochondria were isolated from mouse term placenta using isolation buffer (IB) (200 mM

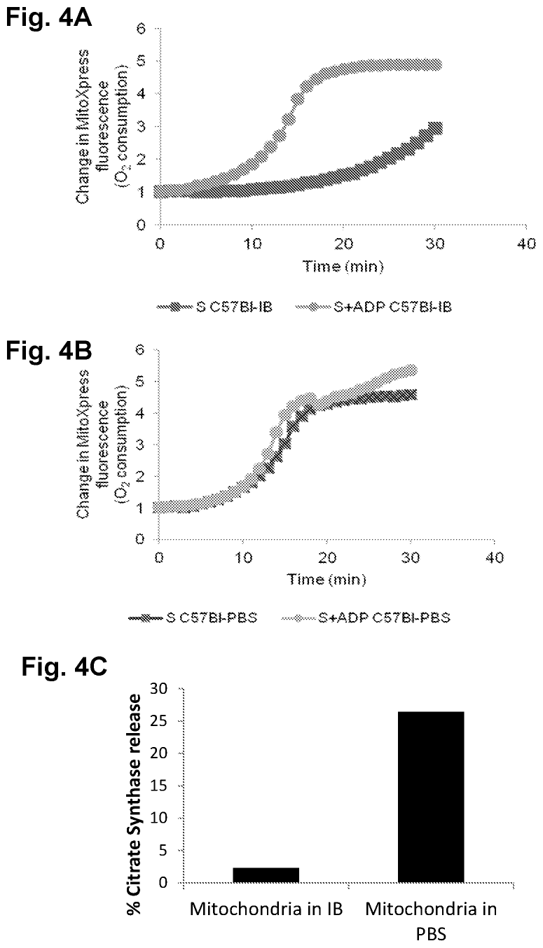

Sucrose, 1 mM EGTA/Tris pH 7.4, 10 mM Tris/Mops pH 7.4 supplemented with 0.2% fatty acid free BSA). The mitochondria pellet was either suspended in IB and incubated on ice, or suspended in PBS and incubated at 37° C. for 10 min. Oxygen consumption was measured for 50 μg mitochondria incubated in the presence of succinate (S) or succinate+ADP (S+A) using the MitoXpress fluorescence probe (Luxcel). As can be seen in FIGS. 4A and 4B, mitochondria that have been incubated with PBS (4B) show oxygen consumption corresponding to un-coupled mitochondria, while mitochondria incubated in IB (4A) show oxygen consumption corresponding to coupled mitochondria.

Mitochondrial inner membrane integrity of mitochondria incubated in IB was compared to that of mitochondria incubated in PBS by measuring citrate synthase release using the CS0720 kit (Sigma). FIG. 4C shows that mitochondria that were incubated in PBS have decreased membrane integrity, as witnessed by citrate synthase release.

### Example 5

### Comparison of Oxygen Consumption and Membrane Integrity of Mitochondria Incubated in Isolation Buffer vs. Mitochondria Incubated in Cell Culture Medium

Mitochondria were isolated from mouse term placenta using isolation buffer (IB) (200 mM Sucrose, 1 mM EGTA/Tris pH 7.4, 10 mM Tris/Mops pH 7.4 supplemented with 0.2% fatty acid free BSA). The mitochondria pellet was suspended for 1 hour at 37° C. either in IB or OptiMEM medium (Gibco).

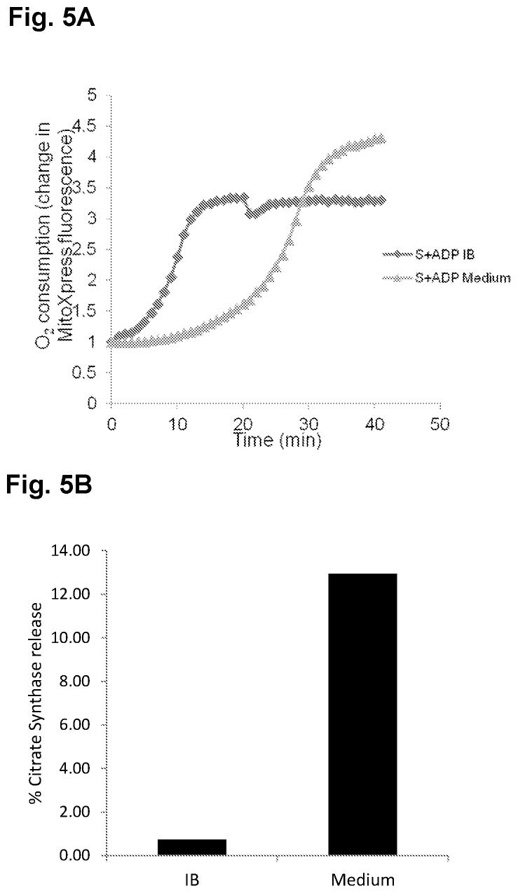

Oxygen consumption was measured for 50 μg mitochondria incubated in the presence of succinate+ADP (S+ADP) using the MitoXpress fluorescence probe (Luxcel). FIG. 5A shows that mitochondria that have been incubated in OptiMEM medium show reduced rate of oxygen consumption relative to mitochondria incubated in IB.

Mitochondrial inner membrane integrity of mitochondria incubated in IB was compared to that of mitochondria incubated in OptiMEM medium by measuring citrate synthase release using the CS0720 kit (Sigma). FIG. 5B shows that mitochondria that were incubated in OptiMEM medium have decreased membrane integrity, as witnessed by citrate synthase release.

### Example 6

### Mitochondria Suspended in a Buffer Containing a High Sucrose Concentration Show Higher Oxygen Consumption

Mitochondria were isolated from human term placenta according to the following protocol:
1. Placenta was rinsed free of blood by using ice-cold IB buffer (isolation buffer: 200 mM sucrose, 1 mM EGTA and 10 mM Tris-MOPS)+0.2% BSA.
2. The placenta was minced into small pieces in 5 ml IB+0.2% BSA using scissors.
3. The suspension was transferred to a 10 ml glass potter and homogenized using a Dounce glass homogenizer by five complete up and down cycles.
4. The homogenate was transferred to a 15 ml tube and centrifuged at 600 g for 10 min at 4° C.
5. The supernatant was transferred to clean centrifuge tubes and the pellet was resuspended in IB buffer, and subjected to a second centrifugation step.
6. The supernatant from steps 4 and 5 was filtered through a 5 μm filter to remove any cells or large cell debris.
7. The supernatant was recovered and centrifuged at 7,000×g for 15 min.
8. The mitochondrial pellet was washed in 10 ml ice cold IB buffer and mitochondria were recovered by centrifugation at 7,000×g for 15 min at 4° C.
9. The supernatant was discarded and the pellet resuspended, containing mitochondria in 200 μl of IB buffer.
10. Protein content was determined by the Bradford assay. Mitoplasts (mitochondria lacking the outer membrane; according to Murthy and Pande, 1987) were prepared by using 10 times diluted IB (20 mM sucrose, 0.1 mM EGTA, 1 mM Tris-MOPS) on the last isolation step (MP). Oxygen consumption over time was measured for 25 μg of mitochondria or mitoplasts using the MitoXpress fluorescence probe (Luxcel) and a Tecan plate reader. The percentage of change in fluorescence was calculated relative to the level of fluorescence at time 0. A trendline was plotted to determine the average change in fluorescence over time which stands for the rate of O₂ consumption (the slope of the line).

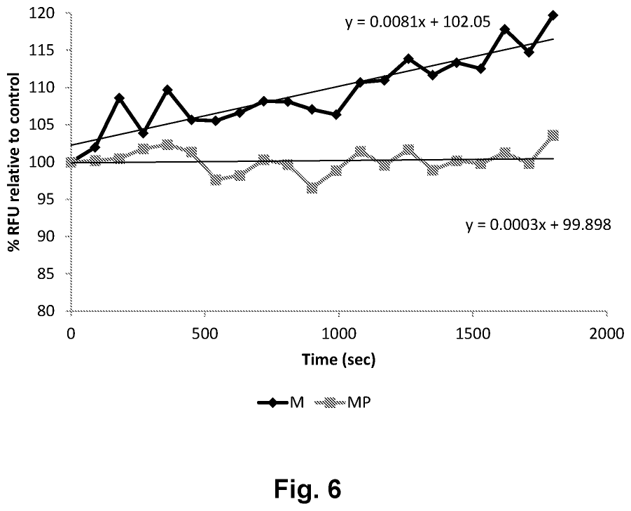

As can be seen in FIG. 6, the rate of oxygen consumption was higher in mitochondria that were suspended in a buffer containing 200 mM sucrose.

### Example 7

### Mouse 3T3 Cells Show Increased Oxygen Consumption Following Treatment with Mitochondria or Mitochondrial Constituents

About 450,000 mouse 3T3 cells were starved for 24 hours in a glucose-free medium containing 5.5 mM D-galactose. Next, the cells were either left untreated (NT), or incubated for 3 hours with either 12.5 μg/ml of mitochondria suspended in isolation buffer and incubated on ice (IB) or 12.5 μg/m1 of mitochondria suspended in PBS, incubated at 37° C. for 10 minutes, frozen and thawed twice and passed through a 25 gauge needle to completely disrupt mitochondrial membranes (PBS).

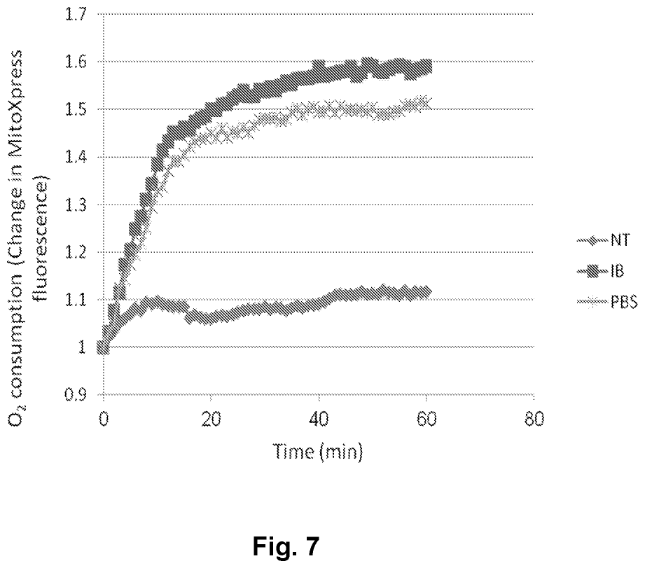

Oxygen consumption of the mouse 3T3 cells was measured using the MitoXpress fluorescence probe (Luxcel). As can be seen in FIG. 7, treatment with either mitochondria incubated in isolation buffer and mitochondrial constituents resulted in an increase in oxygen consumption in mouse 3T3 cells.

### Example 8

### Human 143B Cells Show Increased Levels of Citrate Synthase Activity Following Treatment with Mitochondrial Constituents from Fresh or Frozen Mitochondria

About 60,000 human 143B cells were starved for 72 hours in a glucose-free medium containing 5.5 mM D-galactose and seeded in a 24-wells plate. Next, the cells were incubated for 3 hours with either PBS (NT), 12.5 μg/ml of mitochondria suspended in PBS (PBS) or 12.5 μg/ml of mitochondria suspended in PBS that were frozen for 30 minutes at −80° C. and thawed prior to incubation (PBS frozen). Citrate Synthase activity of human 143B cells was measured using the Citrate Synthase assay kit (Sigma).

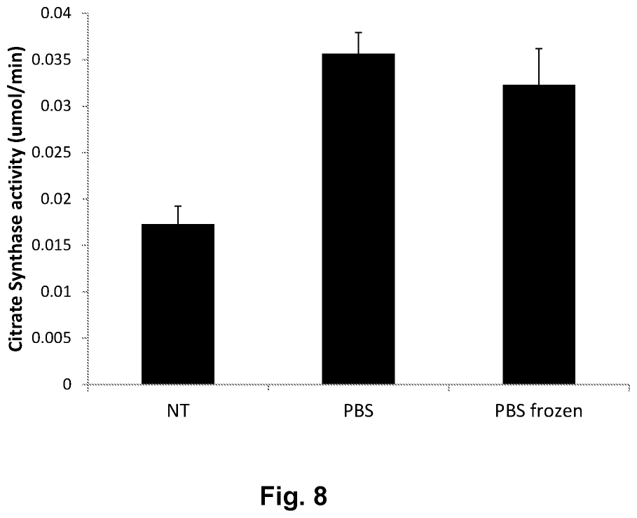

As can be seen in FIG. 8, citrate synthase activity in human 143B increased following treatment with mitochondrial constituents (PBS) or mitochondrial constituents that underwent freezing and thawing (PBS Frozen).

### Example 9

### Mouse Placental Mitochondria Suspended in PBS vs. Isolation Buffer Show Decreased Oxygen Consumption and Increased Citrate Synthase Release

Mitochondria were isolated from mouse term placenta using isolation buffer (IB) (200 mM Sucrose, 1 mM EGTA/Tris pH 7.4, 10 mM Tris/Mops pH 7.4 supplemented with 0.2% fatty acid free BSA). The mitochondria pellet was either suspended in IB and incubated on ice or suspended in PBS and incubated in 37° C. for 10 min. The PBS-suspended mitochondria underwent two cycles of freezing/thawing and were passed through a 25 gauge needle to completely disrupt mitochondrial membranes. The levels of mitochondrial oxygen consumption were measured in 50 μg mitochondria in the presence of succinate (S) or succinate+ADP (S+A) using the MitoXpress fluorescence probe (Luxcel). Mitochondria inner membrane integrity was assessed by measuring the levels of Citrate Synthase release from the mitochondria using the CS0720 kit (Sigma).

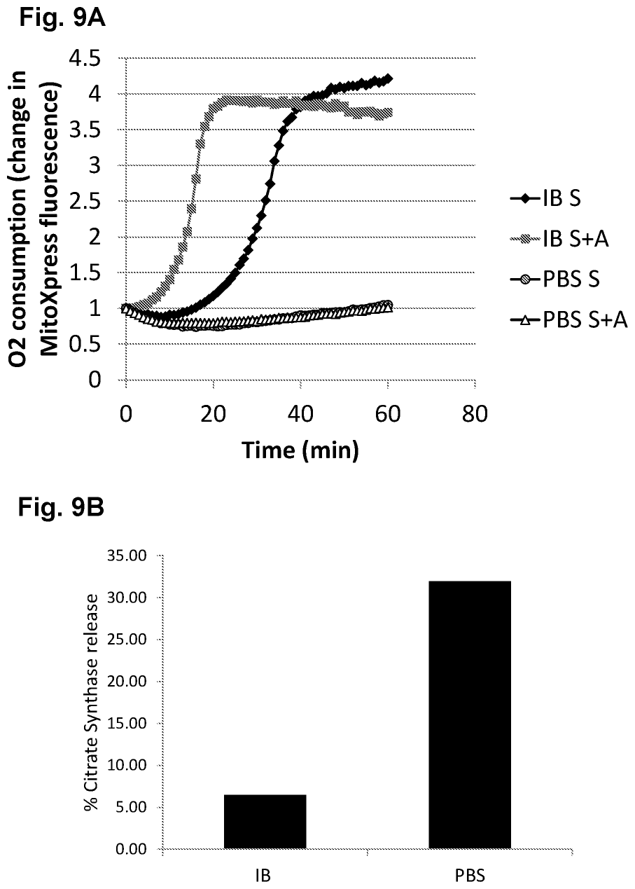

As can be seen in FIG. 9A, mitochondria suspended in PBS show a lower change in oxygen consumption rate. As can be seen in FIG. 9B, mitochondria suspended in PBS show higher release of citrate synthase as compared to mitochondria suspended in IB.

### Example 10

### Human 143B Cells Show Increased Levels of Citrate Synthase Activity Following Incubation with Mitochondria Suspended in Either Isolation Buffer or PBS

About 60,000 human 143B cells were starved for 72 hours in a glucose-free medium containing 5.5 mM D-galactose and seeded in a 24-wells plate. Next, the cells were either left untreated, or incubated for 3 hours with either 12.5 μg/ml of mitochondria suspended in isolation buffer and incubated on ice (IB) or 12.5 μg/ml of mitochondria suspended in PBS, incubated at 37° C. for 10 minutes, frozen and thawed twice and passed through a 25 gauge needle to completely disrupt mitochondrial membranes (PBS). Citrate Synthase activity of human 143B cells was measured using the Citrate Synthase assay kit (Sigma).

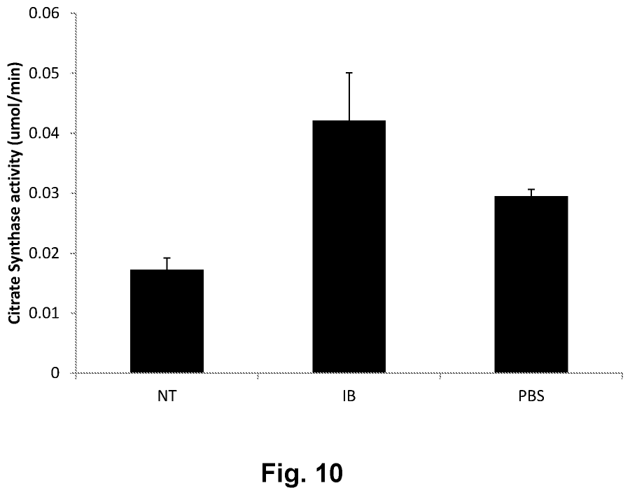

As can be seen in FIG. 10, cells treated with mitochondria that were suspended either in isolation buffer or PBS induced increase in citrate synthase activity of the cells.

### Example 11

### C57BL Mice Show Lower Body Weight and Decreased Cholesterol Levels Following Oral Administration of Mitochondria Isolated from Mung Beans Sprouts

Mitochondria were isolated from mung beans sprouts according to the following protocol:
1. 400 gram of Vigna Radiata sprouts were washed and minced.
2. Homogenization in 2 L of Sucrose Buffer (250 mM Sucrose, 10 Mm Tris/HCl, 1 mM EDTA, pH 7.4).
3. Centrifugation at 600 g, 4° C.
4. Filter by 5μm cutoff.
5. Centrifugation at 8000 g, 4° C.
6. Pellet wash and Centrifugation at 8000 g, 4° C.

4 weeks old male C57BL mice were fed with high fat diet (HFD; 60% fat) or with regular diet for 2 months. Then, mice were treated daily, via oral gavage, with low dose (350 μl, 0.13 μg/μl per day) or high dose (350 μ1, 1.3 μg/μl per day) of mitochondria isolated from mung beans sprouts (n=10 per group). Control mice (n=10) were treated with sucrose buffer as placebo. Mice body weight was recorded at different time points for 27 days after treatment. At the end of experiment, blood was collected from mice in the HFD group, either treated (high dose) or untreated with mitochondria, to assess cholesterol levels.

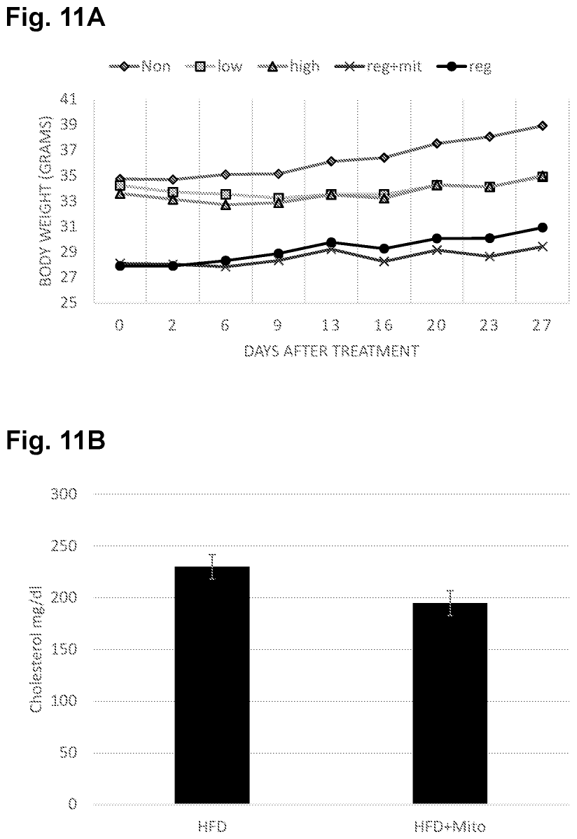

As can be seen in FIG. 11A, in mice fed with high fat diet, both low and high dose mitochondria treatments (marked as ‘low’ and ‘high’) have resulted in reduced weight gain, compared to the control group (non). No significant effect on body weight was observed in mice fed with regular diet, with a similar weight gain in mice treated with high dose mitochondria (reg+mit) compared to control mice (reg). In addition, as can be seen in FIG. 11B, mice in the HFD group showed lower cholesterol levels following treatment with high dose mitochondria (HFD+Mito).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-52. (canceled)

53. A composition comprising intact mitochondria and/or ruptured mitochondria wherein the composition elevates lipid and cholesterol metabolism or induces weight loss or attenuates or reduces weight gain in a subject in need thereof.

54. The composition according to claim 53, wherein the composition comprising the ruptured mitochondria further comprises at least one mitochondrial constituent released from the ruptured mitochondria selected from the group consisting of: mitochondrial protein, mitochondrial nucleic acid, mitochondrial lipid, mitochondrial peptide, mitochondrial saccharide, mitochondrial structure, at least part of a mitochondrial matrix and a combination thereof.

55. The composition according to claim 53, wherein the composition comprising the intact mitochondria further comprises a hypertonic solution.

56. The composition according to claim 53, wherein the mitochondria are:
a) isolated mitochondria, wherein the weight of the mitochondrial proteins in the isolated mitochondria constitutes more than 80% of the combined weight of the mitochondria and other sub-cellular cellular proteins;
b) partially purified mitochondria, wherein the weight of the mitochondrial proteins in the partially purified mitochondria constitutes between 10%-80% of the combined weight of the mitochondria and other sub-cellular proteins; or
c) partially purified mitochondria, wherein the weight of the mitochondrial proteins in the partially purified mitochondria constitutes between 20%-40% of the combined weight of the mitochondria and other sub-cellular proteins.

57. The composition according to claim 53, wherein said mitochondria have undergone a freeze-thaw cycle.

58. The composition according to claim 53, wherein said mitochondria are derived from a cell or a tissue selected from the group consisting of: placenta, placental cells grown in culture, blood cells, plant tissue, plant cells or plant cells grown in culture.

59. The composition according to claim 53, wherein elevating lipid and cholesterol metabolism comprising lowering of least one parameter selected from the group consisting of: blood concentration of total cholesterol, blood concentration of LDL cholesterol, blood concentration of triglycerides, concentration of fatty acids and/or triglycerides in adipose cells, or any combination thereof.

60. A method for elevating lipid and cholesterol metabolism or for inducing weight loss or attenuating or reducing weight gain in a subject in need thereof, the method comprising:
a) providing a composition comprising intact mitochondria and/or ruptured mitochondria; and
b) administering to the subject a therapeutically effective amount of the composition, thereby elevating lipid or cholesterol metabolism.

61. The method according to claim 60, wherein the composition comprising the ruptured mitochondria further comprises at least one mitochondrial constituent released from the ruptured mitochondria.

62. The method according to claim 60, wherein the composition comprising the intact mitochondria further comprises a hypertonic solution.

63. The method according to claim 60, wherein the mitochondria are:
a) isolated mitochondria, wherein the weight of the mitochondrial proteins in the isolated mitochondria constitutes more than 80% of the combined weight of the mitochondria and other sub-cellular cellular proteins;
b) partially purified mitochondria, wherein the weight of the mitochondrial proteins in the partially purified mitochondria constitutes between 10%-80% of the combined weight of the mitochondria and other sub-cellular proteins; or
c) partially purified mitochondria, wherein the weight of the mitochondrial proteins in the partially purified mitochondria constitutes between 20%-40% of the combined weight of the mitochondria and other sub-cellular proteins.

64. The method according to claim 60, wherein said mitochondria have undergone a freeze-thaw cycle.

65. The method according to claim 60, wherein said mitochondria are derived from a cell or a tissue selected from the group consisting of: placenta, placental cells grown in culture, blood cells, plant tissue, plant cells or plant cells grown in culture.

66. The method according to claim 60, wherein said composition is administered to the subject in need thereof by a route selected from the group consisting of: enteral, parenteral, intravenous, intraarterial, subcutaneous, oral and via direct injection into a tissue or an organ, wherein direct injection into a tissue or an organ is administration to adipose tissue.

67. The method according to claim 60, wherein the method further comprises administering a pharmacotherapy, wherein said pharmacotherapy is selected from the group consisting of: drugs that reduce fat absorption, drugs that regulate satiety, drugs for reducing the level of total and LDL cholesterol and any combination thereof

68. A method of treating or preventing a disease in a subject in need thereof, the method comprising:
a) providing a composition comprising intact mitochondria and/or ruptured mitochondria; and
b) administering to the subject a therapeutically effective amount of the composition, thereby elevating lipid or cholesterol metabolism.

69. The method of claim 68, wherein the disease or disorder is selected from the group consisting of obesity, a disease associated with increase in intraperitoneal adipose tissue, visceral obesity, visceral adipose tissue syndrome, fatty liver disease and cellulite.

70. The method according to claim 68, wherein the composition comprising the ruptured mitochondria further comprises at least one mitochondrial constituent released from the ruptured mitochondria selected from the group consisting of: mitochondrial protein, mitochondrial nucleic acid, mitochondrial lipid, mitochondrial peptide, mitochondrial saccharide, mitochondrial structure, at least part of a mitochondrial matrix and a combination thereof.

71. The method according to claim 68, wherein the composition comprising the intact mitochondria further comprises a hypertonic solution.

72. The method according to claim 68, wherein the mitochondria are:
a) isolated mitochondria, wherein the weight of the mitochondrial proteins in the isolated mitochondria constitutes more than 80% of the combined weight of the mitochondria and other sub-cellular cellular proteins;
b) partially purified mitochondria, wherein the weight of the mitochondrial proteins in the partially purified mitochondria constitutes between 10%-80% of the combined weight of the mitochondria and other sub-cellular proteins; or
c) partially purified mitochondria, wherein the weight of the mitochondrial proteins in the partially purified mitochondria constitutes between 20%-40% of the combined weight of the mitochondria and other sub-cellular proteins.

73. The method according to claim 68, wherein said mitochondria have undergone a freeze-thaw cycle.

74. The method according to claim 68, wherein said mitochondria are derived from a cell or a tissue selected from the group consisting of: placenta, placental cells grown in culture, blood cells, plant tissue, plant cells or plant cells grown in culture.

75. The method according to claim 68, wherein said composition is administered to the subject in need thereof by a route selected from the group consisting of: enteral, parenteral, intravenous, intraarterial, subcutaneous, oral and via direct injection into a tissue or an organ.
